SEQUENCE LISTING (63 sequences)

SEQ ID NO: 1 - DNA, 215 bp, Danio rerio
atggccaccaacaacacagctagcatcgctcaggctcgcaactggtggagcagctcaagatggaggccaatattgacagaataaaggtctccaaagcagcagcagatctgacgtcgtactgcgaagcccatgccaaggaggaccccctgctgtcgcccgtgcctgcatccgaaaaccccttcagggagaagaagttcttctgtgccatcctgtaa

SEQ ID NO: 2 - protein, 71 aa, Danio rerio
MATNNTASIAQARKLVEQLKMEANIDRIKVSKAAADLTSYCEAHAKEDPLLSPVPASENPFREKKFFCAIL

SEQ ID NO: 3 - protein, 67 aa, Artificial
  Consensus Sequence
MANNTASIAQARKLVEQLKMEANIDRIKVSKAAADLYCEAHAKEDPLLPVPASENPFREKKFFCAIL

SEQ ID NO: 4 - protein, 71 aa, Homo sapiens
MASNNTASIAQARKLVEQLKMEANIDRIKVSKAAADLMAYCEAHAKEDPLLTPVPASENPFREKKFFCAIL

SEQ ID NO: 5 - protein, 71 aa, Mus musculus; identical to SEQ ID NO: 4

SEQ ID NO: 6 - protein, 71 aa, Artificial
  Consensus Sequence
MAXNNTASIAQARKLVEQLKMEANIDRIKVSKAAADLXXYCEAHAKEDPLLTPVPASENPFREKKFFCAIL

SEQ ID NO: 7 - DNA, 601 bp, Danio rerio
ctcagtgcaagaacctcagtgctgagctgcacatgcatgagggagccaaagagcagaaattacacaccgctattgaagcacaagaccaaaagcacttggaaatgctctgactgacgaggcagcagaggctgaagacgcaccacaggaggtgagctcacttctgaagatcaacaaaacgggcagaagacaaaaagagtcgaaaataagattccctgaatcagctgtcccatctgcgttccagtgtaaccggcctgaaccgccagcctcatggccaccaacaacacagctagcatcgctcaggctcgcaaactggtggagcagctcaagatggaggccaatattgacagaataaaggtctccaaagcagcagcagatctgacgtcgtactgcgaagcccatgccaaggaggaccccctgctgtcgcccgtgcctgcatccgaaaaccccttcagggagaagaagttcttctgtgccatcctgtaacccgcgctgccagccatggactcacctgctccctctgggaaacactcacggacataaaaacacgttgatcttttacaagggctggagagatgagctggagagaagccttcagttctcc

SEQ ID NO: 8 - DNA, 23 bp, Danio rerio
gccatgaggctggcggttcaggc

SEQ ID NO: 9 - DNA, 23 bp, artificial
  Oligonucleotide Morpholino
gcctgaaccgccagcctcatggc

SEQ ID NO: 10 - DNA, 23 bp, artificial
  Oligonucleotide Morpholino
gccatgaggctggcggttcaggc

SEQ ID NO: 11 - DNA, 601 bp, Danio rerio; identical to SEQ ID NO: 7

SEQ ID NO: 12 - DNA, 25 bp, Artificial
  Oligonucleotide Morpholino
tatgctctttctgacctttattctg

SEQ ID NO: 13 - DNA, 361 bp, Danio rerio
ggccaatattgacagaataaaggtcagaaagagcatataatttcctccaaatcctcatactgactagaattgaggtaaatttggttttatatacgcacatacagactttcttgatgttgtatcagaaaagtgttcaaattataaatagggaaattatatcagcagccaatgaccatcaaagttgtaaaatagtgctcatgttatattaaagtcattgcatgcaatgttcaaaaatgacttcaaaaataaatgaatgttcgaatgtagaaccaactttaccccaatctaacaagaaataagcattgtttttgtttactgtatgtgtttatcaacctcaggtctccaaagcagcagcagatct

SEQ ID NO: 14 - protein, 34 aa, Danio rerio
MATNNTASIAQARKLVEQLKMEANIDRIKVRKSI

SEQ ID NO: 15 - DNA, 219 bp, Danio rerio
atggccaccaacaacacagctagcatcgctcaggctcgcaaactggtggagcagctcaagatggaggccaatattgacagaataaaggtctccaaagcagcagcagatctgacgtcgtactgcgaagcccatgccaaggaggaccccctgctgtcgcccgtgcctgcatccgaaaaccccttcagggagaagaagttcttctgtgccatcctgtaactc

SEQ ID NO: 16 - DNA, 271 bp, Danio rerio
atggccaccaacaacacagctagcatcgctcaggctcgcaaactggtggagcagctcaagatggaggccaatattgacagaataaaggtcagaaagagcatataatttcctccaaatcctcatactgactagaattgaggtctccaaagcagcagcagatctgacgtcgtactgcgaagcccatgccaaggaggaccccctgctgtcgcccgtgcctgcatccgaaaaccccttcagggagaagaagttcttctgtgccatcctgtaactc

SEQ ID NO: 17 - protein, 34 aa, Danio rerio; identical to SEQ ID NO: 14

SEQ ID NO: 18 - protein, 74 aa, Homo sapiens
MPVINIEDLTEKDKLKMEVDQLKKEVTLERMLVSKCCEEVRDYVEERSGEDPLVKGIPEDKNPFKELKGGCVIS

SEQ ID NO: 19 - protein, 73 aa, Homo sapiens
MPALHIEDLPEKEKLKMEVDQLRKEVKLQRQQVSKCSEEIKNYIEERSGEDPLVKGIPEDKNPFKEKGSCVIS

SEQ ID NO: 20 - protein, 69 aa, Homo sapiens
MAQDLSEKDLLKMEVDQLKKEVKNTRIPISKAGKEIKEYVEAQAGNDPFLKGIPEDKNPFKEKGGCLIS

SEQ ID NO: 21 - protein, 69 aa, Homo sapiens
MAQDLSEKDLLKMEVEQLKKEVKNTRIPISKAGKEIKEYVEAQAGNDPFLKGIPEDKNPFKEKGGCLIS

SEQ ID NO: 22 - protein, 75 aa, Homo sapiens
MKGETPVNSTMSIGQARKMVEQLKIEASLCRIKVSKAAADLMTYCDAHACEDPLITPVPTSENPFREKKFFCALL

SEQ ID NO: 23 - protein, 75 aa, Homo sapiens
MKEGMSNNSTTSISQARKAVEQLKMEACMDRVKVSQAAADLLAYCEAHVREDPLIIPVPASENPFREKKFFCTIL

SEQ ID NO: 24 - protein, 70 aa, Homo sapiens
MSNNMAKIAEARKTVEQLKLEVNIDRMKVSQAAAELLAFCETHAKDDPLVTPVPAAENPFRDKRLFCVLL

SEQ ID NO: 25 - protein, 68 aa, Homo sapiens
MSATNNIAQARKLVEQLRIEAGIERIKVSKAASDLMSYCEQHARNDPLLVGVPASENPFKDKKPCIIL

SEQ ID NO: 26 - protein, 72 aa, Homo sapiens
MSSKTASTNNIAQARRTVQQLRLEASIERIKVSKASADLMSYCEEHARSDPLLIGIPTSENPFKDKKTCIIL

SEQ ID NO: 27 - protein, 68 aa, Homo sapiens
MSGSSSVAAMKKVVQQLRLEAGLNRVKVSQAAADLKQFCLQNAQHDPLLTGVSSSTNPFRPQKVCSFL

SEQ ID NO: 28 - protein, 68 aa, Homo sapiens
MSGFSSVAATKKVVQQLQLEAGLNSVKVSQAAADLKQFCLQNAQHDPLLTGVSSSTNPFRPQKVCSFL

SEQ ID NO: 29 - protein, 68 aa, Homo sapiens
MSSGASASALQRLVEQLKLEAGVERIKVSQAAAELQQYCMQNACKDALLVGVPAGSNPFREPRSCALL

SEQ ID NO: 30 - protein, 67 aa, Homo sapiens
MEEWDVPQMKKEVESLKYQLAFQREMASKTIPELLKWIEDGIPKDPFLNPDLMKNNPWVEKGKCTIL

SEQ ID NO: 31 - DNA, 25 bp, Artificial
  Oligonucleotide Morpholino
tatgctctttctgacctttattctg

SEQ ID NO: 32 - DNA, 28 bp, Artificial
  Oligonucleotide primer
atcgatatggccaccaacaacacagcta

SEQ ID NO: 33 - DNA, 22 bp, Artificial
  Oligonucleotide Primer
ttacaggatggcacagaagaac

SEQ ID NO: 34 - DNA, 20 bp, Artificial
  Oligonucleotide Primer
cctcacgatccttctggctt

SEQ ID NO: 35 - DNA, 19 bp, Artificial
  Oligonucleotide Primer
attctgcttcacaatgata

SEQ ID NO: 36 - DNA, 990 bp, Homo sapiens
cagtgtgctggcggcccggcgcgagccggcccggccccggtcgggcctccgaaaccatgaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacaccgcggccgccagcacactg

SEQ ID NO: 37 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 38 - DNA, 570 bp, Homo sapiens
accatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtga

SEQ ID NO: 39 - protein, 188 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDSPRPLCPRCTQHHQRPDPRTCRCRCRRRSFLRCQGRGLELNPDTCRCRKLRR

SEQ ID NO: 40 - DNA, 896 bp, Mus musculus
ccccgcccgcgccatggggctctggctaccgccgccccccacgccgccgggctagggccatgcgggctcccggcgctcgccccccgcgggcaccatgagccccctgctccgtcgcctgctgcttgttgcactgctgcagctggctcgcacccaggcccctgtgtcccagtttgatggccccagccaccagaagaaagtggtgccatggatagacgtttatgcacgtgccacatgccagcccagggaggtggtggtgcctctgagcatggaactcatgggcaatgtggtcaaacaactagtgcccagctgtgtgactgtgcagcgctgtggtggctgctgccctgacgatggcctggaatgtgtgcccactgggcaacaccaagtccgaatgcagatcctcatgatccagtacccgagcagtcagctgggggagatgtccctggaagaacacagccaatgtgaatgcagaccaaaaaaaaaggagagtgctgtgaagccagacagggttgccataccccaccaccgtccccagccccgctctgttccgggctgggactctaccccgggagcatcctccccagctgacatcatccatcccactccagccccaggatcctctgcccgccttgcacccagcgccgtcaacgccctgacccccggacctgccgctgccgctgcagacgccgccgcttcctccattgccaagggcggggcttagagctcaacccagacacctgtaggtgccggaagccgcgaaagtgacaagctgctttccagactccacgggcccggctgcttttatggccctgcttcacagggacgaagagtggagcacaggcaaacctcctcagtctgggaggtcactgccccaggacctggaccttttagagagctct

SEQ ID NO: 41 - protein, 207 aa, Mus musculus
MSPLLRRLLLVALLQLARTQAPVSQFDGPSHQKKVVPWIDVYARATCQPREVVVPLSMELMGNVVKQLVPSCVTVQRCGGCCPDDGLECVPTGQHQVRMQILMIQYPSSQLGEMSLEEHSQCECRPKKKESAVKPDRVAIPHHRPQPRSVPGWDSTPGASSPADIIHPTPAPGSSARLAPSAVNALTPGPAAAAADAAASSIAKGGA

SEQ ID NO: 42 - DNA, 2848 bp, Mus musculus
cgggattgcacggaaacttttcgtccaacttctgggctcttctcgctccgtagtagccgtggtctgcgccgcaggagacaaaccgatcggagctgggagaagtgctagctcgggcctggagaagccggggcccgagaagagaggggaggaagagaaggaagaggagagggggccgcagtgggcgctcggctctcaggagccgagctcatggacgggtgaggcggccgtgtgcgcagacagtgctccagccgcgcgcgcgccccaggccccggcccgggcctcggttccagaagggagaggagcccgccaaggcgcgcaagagagcgggctgcctcgcagtccgagccggagagggagcgcgagccgcgccggccccggacgggcctccgaaaccatgaactttctgctctcttgggtgcactggaccctggctttactgctgtacctccaccatgccaagtggtcccaggctgcacccacgacagaaggagagcagaagtcccatgaagtgatcaagttcatggatgtctaccagcgaagctactgccgtccgattgagaccctggtggacatcttccaggagtaccccgacgagatagagtacatcttcaagccgtcctgtgtgccgctgatgcgctgtgcaggctgctgtaacgatgaagccctggagtgcgtgcccacgtcagagagcaacatcaccatgcagatcatgcggatcaaacctcaccaaagccagcacataggagagatgagcttcctacagcacagcagatgtgaatgcagaccaaagaaagacagaacaaagccagaaaatcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctgcaggaaggagcctccctcagggtttcgggaaccagacctctcaccggaaagaccgattaaccatgtcaccaccacgccatcatcgtcaccgttgacagaacagtccttaatccagaaagcctgacatgaaggaagaggagactcttcgaggagcactttgggtccggagggcgagactccggcagacgcattcccgggcaggtgaccaagcacggtccctcgtgggactggattcgccattttcttatatctgctgctaaatcgccaagcccggaagattagggttgtttctgggattcctgtagacacacccacccacatacacacatatatatatattatatatataaataaatatatatgttttatatataaaatatatatatattcttttttttaaattaactctgctaatgttattggtgtcttcactggatatgtttgactgctgtggacttgtgttgggaggaggatgtcctcactcggatgccgacacgggagacaatgggatgaaaggcttcagtgtggtctgagagaggccgaagtccttttgcctgccggggagcaagcaaggccagggcacgggggcacatttgctcacttccagaaacacgacaaacccattcctggccctgagtcaagaggacagagagacagatgatgacagagaaagagataaagatgccggttccaaccagaagtttggggagcctcaggacatggcatgctttgtggatccccatgatagtctacaaaagcaccccgcccctctgggcactgcctggaagaatcgggagcctggccagccttcagctcgctcctccacttctgaggggcctaggaggcctcccacaggtgtcccggcaagagaagacacggtggtggaagaagaggcctggtaatggcccctcctcctgggaccccttcgtcctctccttaccccacctcctgggtacagcccaggaggaccttgtgtgatcagaccattgaaaccactaattctgtccccaggagacttggctgtgtgtgtgagtggcttacccttcctcatcttcccttcccaaggcacagagcaatggggcaggacccgcaagcccctcacggaggcagagaaaagagaaagtgttttatatacggtacttatttaatagccctttttaattagaaattaaaacagttaatttaattaaagagtagggtttttttcagtattcttggttaatatttaatttcaactatttatgagatgtatctctcgctctctcttatttgtacttgtgtgtgtgtgtgtgtgtgtgtgtgtgtgtgtgtgtgtgtgtgtatgaaatctgtgtttccaatctctctctcccagatcggtgacagtcactagcttgtcctgagaagatatttaattttgctaacactcagctctgccctcccttgtccccaccacacattcctttgaaataaggtttcaatatacatttacatactatatatatatttggcaacttgtgtttgtatataaatatatatatatatatatatgtttatgtatatatgtgattctgataaaatagacattgctattctgttttttatatgtaaaaacaaaacaagaaaaatagagaattctacatactaaatctctctccttttttaattttaatatttgttatcatttatttattggtgctactgtttatccgtaataattgtgggggaaaaagatattaacatcacgtctttgtctctagagcagttttccgagatattccgtagtacatatttatttttaaacagcaacaaagaaatacagatatatcttaaaaaaaaaagcattttgtattaaagaattgaattctgatctcaaaaaaaaaaaaaaaaaaa

SEQ ID NO: 43 - protein, 190 aa, Mus musculus
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIGEMSFLQHSRCECRPKKDRTKPENHCEPCSERRKHLFVQDPQTCKCSCKNTDSRCKARQLELNERTCRCDKPRR

SEQ ID NO: 44 - DNA, 2678 bp, Danio rerio
gttaacatccacggcagtacgaaccagcgcgacgcaaaacagtcacggaaataagacgaggaaaaaacggacgcgtgtgcagcggtcaaaccaaggattgccactggatctctttacagcaagcctttgctcttgatgataatcatatgcgcagaagaagccgtcctctggccctatagacgcgcgtgcacactggaggctgcgcttggaagtggacgaaactttttgtttgttttgcctctgacgttttggattcagattcattaacgtcgctttccgttatactgtggattttaattttaaattacgcagaggaccaagatagcgtggatttccacttttctttatattataattattattgttattcttatttatatcctttttttttctctggaggagcgggtggcggtccagaagttaattttagcggattcgacgcaggtgcttcggatctgaagggcagaacggatgtgttactttgacccacgatatcacactcggttcattcatacccagcagcttcgctttcttaattgtttgagagccagcgactcaccgcaacactccactggaattacaatcatgaacttggttgtttatttgatacagttatttctcgcggctctcctccatctgtctgctgtaaaggctgcccacatacccaaagaagggggaaagagcaaaaatgatgtgattcccttcatggatgtgtataaaaagagtgcgtgcaagacccgagagctgctggtagacatcatccaggagtatcccgatgagatcgagcacacgtacatcccgtcctgtgtggttctcatgcgctgtgcaggatgctgtaatgatgaggcgctcgaatgcgtcccgacagagacacgaaacgtcactatggaggtgctgcgggtcaagcaacgcgtatcgcagcataattttcagctgagtttcacagaacacaccaagtgtgaatgcaggccaaaggcagaagtcaaagcaaagaaagaaaaccactgtgagccttgctcagagagaaggaagcgcttgtatgtgcaggaccccctcacctgtaaatgctcctgcaaattcacacaaatgcaatgcaagtccagacaacttgagttaaacgaaagaacttgcagatgtgaaaagccaagatgatgatcgagcgaacgccgggcagaactctgcatgaaggaaggaacttaaataaaaactttttaaacggacagcactttcaaaacctaaggaagaacattccttggatgaactgcaaacactatcctgaaacgaaacactaagtgcaggagatatcatcccattgtatatttttaagatattaatataaagaatattatatatataaatatatatattaaatgacaatgtgtgtcgccgctatcacctacaaataagtcaacgaagacgctgctttcgcgtgtgaatgggatatattgcagtgtcactacgtctgaggcctgtgattggacgaaacacagcgagggatgcataattaaagactcagccttcactgagagccagacaacctccggacctttctttacgaacagtagcagactgtcaatcaaaggacaggaagtgacacattaaagattcttttccggaatcttcggaacagtgggtccaggagcggaacaacgaatgcggaactatctttttctgtgatgtcacttgctagggtgaattcgtagtcgaaaagacacctgtactgactaacatccaataggacttggtgtgataatactaaagggatattacgtgtatgtatatatgacaaaacacacacacaaatcagtgaacttttgacattccacattaaaaatactatagtaagtagtgtttttgaaccatactgtggtaaagtgtattatactgacgtttttttttgctgcttgttcaaactacttatttaaaattgagttgaaccaacacaattcttaagttattttgggcacaacttaattgttttatctttgatccacttaaatttgtaagttaagttgacttaatcggtttgtgttgggacaacatgaagaaattgtgtggaacccagcctttttcacagtgtatattataatatcaagtactttactatagtgtattccaaaagcgacactgtagaattgactatagtattttgtaatgtgaaggaaacgaagtcgttctgttagtacgcggtattcataaatatatttttttttccaaagaaagagaatattgcacacaactttaggaactggtttattaccacacgatatctgtatatacatttctaatatccctttgtgtagttcgctgcaccttaatttaaattgttagagcagtattctgtctatttaatttgatctatttatatcgtgttattgtttcatgaatctgaaaaactgtggacggctttttctagacagacaaacgagaggcaaaaacattcctgaaaagcgccttttttagtttgtttatatcggcgatcagctcggtggagaagtgttgggatgacaaacaaacattctctttgtgtgccacgttttcagagtaatatatttaatggtgctgttaacatctttttttgtgtgtgtttttagtttattattattccgcatatttatttttaaataatattgttttgtattaaagcagaaatggatttcaaaaaaaaaaaaaaaaaa

SEQ ID NO: 45 - protein, 188 aa, Danio rerio
MNLVVYLIQLFLAALLHLSAVKAAHIPKEGGKSKNDVIPFMDVYKKSACKTRELLVDIIQEYPDEIEHTYIPSCVVLMRCAGCCNDEALECVPTETRNVTMEVLRVKQRVSQHNFQLSFTEHTKCECRPKAEVKAKKENHCEPCSERRKRLYVQDPLTCKCSCKFTQMQCKSRQLELNERTCRCEKPR

SEQ ID NO: 46 - DNA, 2076 bp, Homo sapiens
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 47 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 48 - DNA, 2106 bp, Homo sapiens
caaaaaagtttcaatccagagttgggttccagctttctgtagctgtaagcattggtggccacaccacctccttacaaagcaactagaacctgcggcatacgttggagagatttttttaattttctggacatgaagtaaatttagagtgctttctaatttcaggtagaagacatgtccaccttctgattatttttggagcatgagaacattttgatttttttcatctctctctccccacccctaagattgtgcaaaaaaagcgtaccttgcctaattgaaataatttcattggattttgatcagaactgattatttggttttctgtgtgaagttttgaggtttcaaactttccttctggagaatgccttttgaaacaattttctctagctgcctgatgtcaactgcttagtaatcagtggatattgaaatattcaaaatgtacagagagtgggtagtggtgaatgttttcatgatgttgtacgtccagctggtgcagggctccagtaatgaacatggaccagtgaagcgatcatctcagtccacattggaacgatctgaacagcagatcagggctgcttctagtttggaggaactacttcgaattactcactctgaggactggaagctgtggagatgcaggctgaggctcaaaagttttaccagtatggactctcgctcagcatcccatcggtccactaggtttgcggcaactttctatgacattgaaacactaaaagttatagatgaagaatggcaaagaactcagtgcagccctagagaaacgtgcgtggaggtggccagtgagctggggaagagtaccaacacattcttcaagcccccttgtgtgaacgtgttccgatgtggtggctgttgcaatgaagagagccttatctgtatgaacaccagcacctcgtacatttccaaacagctctttgagatatcagtgcctttgacatcagtacctgaattagtgcctgttaaagttgccaatcatacaggttgtaagtgcttgccaacagccccccgccatccatactcaattatcagaagatccatccagatccctgaagaagatcgctgttcccattccaagaaactctgtcctattgacatgctatgggatagcaacaaatgtaaatgtgttttgcaggaggaaaatccacttgctggaacagaagaccactctcatctccaggaaccagctctctgtgggccacacatgatgtttgacgaagatcgttgcgagtgtgtctgtaaaacaccatgtcccaaagatctaatccagcaccccaaaaactgcagttgctttgagtgcaaagaaagtctggagacctgctgccagaagcacaagctatttcacccagacacctgcagctgtgaggacagatgcccctttcataccagaccatgtgcaagtggcaaaacagcatgtgcaaagcattgccgctttccaaaggagaaaagggctgcccaggggccccacagccgaaagaatccttgattcagcgttccaagttccccatccctgtcatttttaacagcatgctgctttgccaagttgctgtcactgtttttttcccaggtgttaaaaaaaaaaatccattttacacagcaccacagtgaatccagaccaaccttccattcacaccagctaaggagtccctggttcattgatggatgtcttctagctgcagatgcctctgcgcaccaaggaatggagaggaggggacccatgtaatccttttgtttagttttgtttttgttttttggtgaatgagaaaggtgtgctggtcatggaatggcaggtgtcatatgactgattactcagagcagatgaggaaaactgtagtctctgagtcctttgctaatcgcaactcttgtgaattattctgattcttttttatgcagaatttgattcgtatgatcagtactgactttctgattactgtccagcttatagtcttccagtttaatgaactaccatctgatgtttcatatttaagtgtatttaaagaaaataaacaccattattcaagccaaaaaaaaaaaaaaaaaaaaaaaaaaaaaaaaaaaaaaaaaaaaaaaaaaaaaaaaaaaaaaaaaaaa

SEQ ID NO: 49 - protein, 354 aa, Homo sapiens
MYREWVVVNVFMMLYVQLVQGSSNEHGPVKRSSQSTLERSEQQIRAASSLEELLRITHSEDWKLWRCRLRLKSFTSMDSRSASHRSTRFAATFYDIETLKVIDEEWQRTQCSPRETCVEVASELGKSTNTFFKPPCVNVFRCGGCCNEESLICMNTSTSYISKQLFEISVPLTSVPELVPVKVANHTGCKCLPTAPRHPYSIIRRSIQIPEEDRCSHSKKLCPIDMLWDSNKCKCVLQEENPLAGTEDHSHLQEPALCGPHMMFDEDRCECVCKTPCPKDLIQHPKNCSCFECKESLETCCQKHKLFHPDTCSCEDRCPFHTRPCASGKTACAKHCRFPKEKRAAQGPHSRKNP

SEQ ID NO: 50 - DNA, 1818 bp, Mus musculus
gcaaaagttgcgagccgccgagtcccgggagacgctcgcccaggggggtccccgggaggaaaccacgggacagggaccaggagaggacctcagcctcacgccccagcctgcgccagccaacggaccggcctccctgctcccggtccatccaccatgcacttgctgtgcttcttgtctctggcgtgttccctgctcgccgctgcgctgatccccagtccgcgcgaggcgcccgccaccgtcgccgccttcgagtcgggactgggcttctcggaagcggagcccgacgggggcgaggtcaaggcttttgaaggcaaagacctggaggagcagttgcggtctgtgtccagcgtagatgagctgatgtctgtcctgtacccagactactggaaaatgtacaagtgccagctgcggaaaggcggctggcagcagcccaccctcaataccaggacaggggacagtgtaaaatttgctgctgcacattataacacagagatcctgaaaagtattgataatgagtggagaaagactcaatgcatgccacgtgaggtgtgtatagatgtggggaaggagtttggagcagccacaaacaccttctttaaacctccatgtgtgtccgtctacagatgtgggggttgctgcaacagcgaggggctgcagtgcatgaacaccagcacaggttacctcagcaagacgttgtttgaaattacagtgcctctctcacaaggccccaaaccagtcacaatcagttttgccaatcacacttcctgccggtgcatgtctaaactggatgtttacagacaagttcattcaattattagacgttctctgccagcaacattaccacagtgtcaggcagctaacaagacatgtccaacaaactatgtgtggaataactacatgtgccgatgcctggctcagcaggattttatcttttattcaaatgttgaagatgactcaaccaatggattccatgatgtctgtggacccaacaaggagctggatgaagacacctgtcagtgtgtctgcaagggggggcttcggccatctagttgtggaccccacaaagaactagatagagactcatgtcagtgtgtctgtaaaaacaaacttttccctaattcatgtggagccaacagggaatttgatgagaatacatgtcagtgtgtatgtaaaagaacgtgtccaagaaatcagcccctgaatcctgggaaatgtgcctgtgaatgtacagaaaacacacagaagtgcttccttaaagggaagaagttccaccatcaaacatgcagttgttacagaagaccgtgtgcgaatcgactgaagcattgtgatccaggactgtcctttagtgaagaagtatgccgctgtgtcccatcgtattggaaaaggccacatctgaactaagatcataccagttttcagtcagtcacagtcatttactctcttgaagactgttggaacagcacttagcactgtctatgcacagaaagactctgtgggaccacatggtaacagaggcccaagtctgtgtttattgaaccatgtggattactgcgggagaggactggcactcatgtgcaaaaaaaacctcttcaaagactggttttctgccagggaccagacagctgaggtttttctcttgtgatttaaaaaaagaatgactatataatttatttccactaaaaatattgttcctgcattcatttttatagcaataacaattggtaaagctcactgtgatcagtatttttataacatgcaaaactatgtttaaaataaaatgaaaattgtattataaaaaaaaaaaaaaaaaaaaaaaaa

SEQ ID NO: 51 - protein, 415 aa, Mus musculus
MHLLCFLSLACSLLAAALIPSPREAPATVAAFESGLGFSEAEPDGGEVKAFEGKDLEEQLRSVSSVDELMSVLYPDYWKMYKCQLRKGGWQQPTLNTRTGDSVKFAAAHYNTEILKSIDNEWRKTQCMPREVCIDVGKEFGAATNTFFKPPCVSVYRCGGCCNSEGLQCMNTSTGYLSKTLFEITVPLSQGPKPVTISFANHTSCRCMSKLDVYRQVHSIIRRSLPATLPQCQAANKTCPTNYVWNNYMCRCLAQQDFIFYSNVEDDSTNGFHDVCGPNKELDEDTCQCVCKGGLRPSSCGPHKELDRDSCQCVCKNKLFPNSCGANREFDENTCQCVCKRTCPRNQPLNPGKCACECTENTQKCFLKGKKFHHQTCSCYRRPCANRLKHCDPGLSFSEEVCRCVPSYWKRPHLN

SEQ ID NO: 52 - DNA, 1581 bp, Mus musculus
  misc_feature (1407)..(1407): n is a, c, g, or t
ttccgggctttgctggagaatgccttttgcaacacttttcagtagctgcctggaaacaactgcttagtcatcggtagacatttaaaatattcaaaatgtatggagaatggggaatggggaatatcctcatgatgttccatgtgtacttggtgcagggcttcaggagcgaacatggaccagtgaaggatttttcttttgagcgatcatcccggtccatgttggaacgatctgaacaacagatccgagcagcttctagtttggaggagttgctgcaaatcgcgcactctgaggactggaagctgtggcgatgccggttgaagctcaaaagtcttgccagtatggactcacgctcagcatcccatcgctccaccagatttgcggcaactttctatgacactgaaacactaaaagttatagatgaagaatggcagaggacccaatgcagccctagagagacatgcgtagaagtcgccagtgagctggggaagacaaccaacacattcttcaagcccccctgtgtaaatgtcttccggtgtggaggctgctgcaacgaagagggtgtgatgtgtatgaacacaagcacctcctacatctccaaacagctctttgagatatcagtgcctctgacatcagtgcccgagttagtgcctgttaaaattgccaaccatacgggttgtaagtgcttgcccacgggcccccgccatccttactcaattatcagaagatccattcagaccccagaagaagatgaatgtcctcattccaagaaactctgtcctattgacatgctgtgggataacaccaaatgtaaatgtgttttgcaagacgagactccactgcctgggacagaagaccactcttacctccaggaacccactctctgtggaccgcacatgacgtttgatgaagatcgctgtgagtgcgtctgtaaagcaccatgtccgggagatctcattcagcacccggaaaactgcagttgctttgagtgcaaagaaagtctggagagctgctgccaaaagcacaagatttttcacccagacacctgcagctgtgaggacagatgtccttttcacaccagaacatgtgcaagtagaaagccagcctgtggaaagcactggcgctttccaaaggagacaagggcccagggactctacagccaggagaacccttgattcaacttcctttcaagtccccccatctctgtcattttaaacagctcactgctttgtcaagttgctgtcactgttgcccactacccctgcccccccccccccccgcctccaggtgttagaaaagttgatttgacctagtgtcatggtaaagccacatttccatgcaatggcggctaggtgattccccagttcactgacaaatgacttgtagcttcaaatgtctttgcgccatcancactcaaaaaggaaggggtctgaagaaccccttgtttgataaataaaaacaggtgcctgaaacaaaatattaggtgccactcgattgggtccctcgggctggccaaattccaagggcaatgctcctgaatttattgtgccccttccttaatgcggaatttccttttgtttgatt

SEQ ID NO: 53 - protein, 358 aa, Mus musculus
MYGEWGMGNILMMFHVYLVQGFRSEHGPVKDFSFERSSRSMLERSEQQIRAASSLEELLQIAHSEDWKLWRCRLKLKSLASMDSRSASHRSTRFAATFYDTETLKVIDEEWQRTQCSPRETCVEVASELGKTTNTFFKPPCVNVFRCGGCCNEEGVMCMNTSTSYISKQLFEISVPLTSVPELVPVKIANHTGCKCLPTGPRHPYSIIRRSIQTPEEDECPHSKKLCPIDMLWDNTKCKCVLQDETPLPGTEDHSYLQEPTLCGPHMTFDEDRCECVCKAPCPGDLIQHPENCSCFECKESLESCCQKHKIFHPDTCSCEDRCPFHTRTCASRKPACGKHWRFPKETRAQGLYSQENP

SEQ ID NO: 54 - DNA, 25 bp, Artificial
  Oligonucleotide Morpholino
gtatcaaataaacaaccaagttcat

SEQ ID NO: 55 - DNA, 25 bp, Artificial
  Oligonucleotide Morpholino
gtaacaattaaacaaccatgttgat

SEQ ID NO: 56 - DNA, 25 bp, Artificial
  Oligonucleotide Morpholino
taagaaagcgaagctgctgggtatg

SEQ ID NO: 57 - DNA, 456 bp, Bos taurus
gtgcaggcgagctccggcgcacgcgcccagcccagccccgggagccaagccagatctgccagtgagcctcaggctttgggaattgaagagtgtatctgaaacctacccagcattccaatggccagcaacaacaccgccagcatagcacaagccaggaaactggtagaacagctgaagatggaagccaacatcgataggataaaggtgtccaaggcagctgcagatttgatggcctactgtgaagcgcatgccaaggaagatcccctcctgacacctgttccggcttcagaaaacccatttagggagaagaagtttttctgtgctatcctttaagtcttcaggaggaacctgaggagcctcggggctccagggacactgatgtagagtttttagcaaagtgggcgcctttctagtccacagcatttaaagagagggaggagaaccatcctggagtct

SEQ ID NO: 58 - protein, 71 aa, Bos taurus; identical to SEQ ID NO: 4

SEQ ID NO: 59 - DNA, 203 bp, Mus musculus
ttcaggtgtccaaggcagctgctgacttgatggcctactgtgaggcacatgccaaggaagaccctctgctgaccccagtcccagcctcagaaaacccctttcgggagaagaagttcttctgcgccatcctttaagtctctgagaggaggctgaagagtgttggggctcctgggacatgtagagttcctagcaaagtgggcgcc

SEQ ID NO: 60 - DNA, 216 bp, Homo sapiens
atggccagcaacaacaccgccagcatagcacaagccaggaagctggtagagcagcttaagatggaagccaatatcgacaggataaaggtgtccaaggcagctgcagatttgatggcctactgtgaagcacatgccaaggaagaccccctcctgacccctgttccggcttcagaaaacccgtttagggagaagaagtttttctgtgccatcctttaa

SEQ ID NO: 61 - DNA, 1145 bp, Mus musculus
ggagccaagcaagtcagatctgccagggagcgtcaggccttgaacactgaccagagtctctgaagaccccatccaacgctccaatggccagcaacaacaccgccagcatagcacaagccaggaagctggtagaacagctgaagatggaagccaacatcgacaggataaaggtgtccaaggcagctgctgacttgatggcctactgtgaggcacatgccaaggaagaccctctgctgaccccagtcccagcctcagaaaacccctttcgggagaagaagttcttctgcgccatcctttaagtctctgagaggaggctgaagagtgttggggctcctgggacatacatgtagagttcctagcaaagtgggcgcctttctcgtccacagcatttaaagagaggaaggagaaccatcctggacactccgggctgtgcatgtttaaagaaatgtccccttatgagaatgaaagctgattccgtgtcccaactttagagatctgaccctgcagaccggcctggaggagggaaatgtataaaaaatgagaatggtaatcacttcttttctgctgtccctctaagacattttctgcttcatatttataaacaaaaataaaacatttaaaagcccggtgttcctggagcctgagaatatgggctggacatttctgggtaatgagaaattggtcttttcaaaatacccctctgataagctggtcgcccccatgctagcccacaaatccaagtttaatggggtttccaacgtttaagtcttttggtgcctttttcttttccttccctttcttctcttttagagggctggagaggataagactgggtttgtgtgttagtctcaactccaagcaggaatgaaccctgagatgtttagaaaagtgttccccatggggatttcttcatgagaataacaagaaaatagaaagaaatacatacatgtacacacacacacatgcacgcacacacacacaccacacaattccggctctctgtgttctcttaccactattcttaatatttgtatatgacagttttgattctacgagttaaagtgaaccacgtgttgtgactggtgcttccatatctgtgatagtttgtgagtactattgcatgaacatgtccctacatcgcatcc

SEQ ID NO: 62 - DNA, 20 bp, Artificial
  Oligonucleotide Primer
ggagccaagcaagtcagatc

SEQ ID NO: 63 - DNA, 20 bp, Artificial
  Oligonucleotide Primer
ggatgcgatgtagggacatg

(19) United States
(12) Patent Application Publication
(10) Pub. No.: US 2006/0154285 A1
(43) Pub. Date: Jul. 13, 2006

(54) Zebrafish heterotrimer G-protein gamma 2 subunit (GNG2)

(76) Inventors: Janet D. Robishaw, Catawissa, PA (US); Tinchung Leung, Danville, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/320,422

(22) Filed: Dec. 28, 2005

Related U.S. Application Data
(60) Provisional application No. 60/640,802, filed on Dec. 29, 2004.

(51) Int. Cl. C12Q 1/68; C07H 21/04; C12P 21/06; A61K 48/00; C07K 14/82; A61K 39/395; C07K 16/30

(52) U.S. Cl. 435/6; 435/69.1; 435/320.1; 435/325; 530/350; 530/388.8; 514/44; 536/23.5

(57) ABSTRACT

The invention provides sequences of the zebrafish heterotrimeric G-protein gamma 2 subunit (GNG2). The invention also provides methods of inhibiting and promoting GNG2-dependent angiogenesis in vertebrates, particular mammals, including humans, for the treatment of angiogenesis-related diseases. The invention also provides methods of identifying compounds that promote or inhibit angiogenesis through their interaction with GNG2. The invention further provides methods of modulating angiogenesis through the modulation of both GNG2 and VEGF.

### CROSS-REFERENCE TO RELATED APPLICATIONS

This Application claims benefit of U.S. Provisional Application No. 60/640,802, filed Dec. 29, 2004, the disclosure of which is hereby incorporated by reference in its entirety.

### REFERENCE TO GOVERNMENT GRANTS

Portions of the disclosure herein may have been supported in part by the National Institutes of Health Grant No. GM 58191. The United States Government may have certain rights in this application.

### FIELD OF THE INVENTION

The invention relates to vertebrate heterotrimer G-protein gamma 2 subunit (GNG2), including human, mouse, zebrafish and other vertebrates which may be derived from any species, as a drug target for anti-angiogenesis and pro-angiogenesis related therapies.

### BACKGROUND OF THE INVENTION

Angiogenesis is a process where new blood vessels are formed from pre-existing blood vessels. This involves proliferation, differentiation and migration of endothelial cells and possibly other cell types found in the vasculature, such as smooth muscle cells and fibroblasts. Alteration of this process, either by potentiation or by inhibition, can be beneficial for the treatment of human diseases, such as cancer, macular degeneration, rheumatoid arthritis, Alzheimer's disease, wound healing, atherosclerosis and ischemia.

Inhibition of angiogenesis represents a powerful new approach to cancer therapy. To fully realize the potential of this avenue for cancer treatment, assays that can rapidly screen compounds for anti-angiogenic activity are essential. Solid tumors require adequate supply of nutrients from the blood to survive, grow, and metastasize (Hanahan, D. & J. Folkman (1996) Cell 86:353-364; Li, C. Y. et al. (2000) Cancer Met. Rev. 19:7-11). New blood vessels that nourish growing tumors by sprouting from existing blood vessels, a process known as angiogenesis. In recent years, angiogenesis has received considerable attention as a novel process to target against cancer. Many drugs already in clinical trials have been shown to have anti-angiogenic activity and new drugs are being developed specifically for their ability to stop such blood vessel growth (Rosen, L. (2000) Oncologist 5(suppl. 1):20-27).

Anti-angiogenic drugs have had mixed success in clinical application. Many new compounds may need to be tested in vivo to identify drugs capable of treating a wide range of tumors. Thus, suitable in vivo assays for screening potential anti-angiogenic compounds are increasingly important. U.S. Patent Publication No. 20040143865 to Rubinstein et al. provides an assay using the zebrafish (Danio rerio) that provides the relevance of an in vivo environment as well as the potential for high-throughput drug screening. The technique involves generating a transgenic line of zebrafish that expresses a reporter protein, for example, green reef coral fluorescent protein (G-RCFP, Matz, M. V. et al. (2000) Nat. Biotechnol. 17:969-973) or red fluorescent protein (dsRed2) which are specifically expressed in the blood vessels.

The zebrafish has become a well-accepted model for studies of vertebrate development. Unlike the mouse, zebrafish embryos develop outside the mother and are transparent, facilitating the observation of differentiating tissues and organs. The vascular system of the zebrafish, in particular, has been well described and shown to be highly conserved from the zebrafish to human (Isogai, S. et al. (2001) Dev. Biol. 230(2):278-301; Vogel, A. M. & B. M. Weinstein (2000) Trends Cardiovasc. Med. 10(8):352-360). Furthermore, zebrafish embryos can live for several days without a significant blood supply, thus facilitating the study of embryos with vascular defects. The intersegmental blood vessels in the zebrafish embryos form by angiogenic sprouting which is very similar to tumor angiogenesis and this process appears to require the same proteins shown to be necessary for blood vessel growth in mammals. For instance, the anti-angiogenic compound PTK787/ZK222584, an inhibitor of vascular endothelial growth factor (VEGF) receptor tyrosine kinases, has been shown to affect the formation of zebrafish blood vessels (Chan, J. et al. (2002) Cancer Cell 1:257-267). Current methods of visualizing blood vessels in the zebrafish include whole mount in situ hybridization of vascular endothelial cell marker (Fouquet, B. et al. (1997) Dev. Biol. 183:37-48.; Liao, W. et al. (1997) Development 124:381-389), detection of endogenous alkaline phosphatase activity in the vessels and microangiography of the circulating cardiovascular system. The latter technique involves injection of fluorescent beads into the circulation of living zebrafish larvae (Weinstein, B. M. et al. (1996) Nat. Med. 1:1143-1147) and is useful for visualization of blood vessels in a complete circulatory system. It was found that the formation of intersegmental blood vessels, blocked by application of tyrosine kinase inhibitors that target the VEGF receptor, could be easily visualized in the transgenic fish expressing the fluorescent protein in the vascular system. Heterotrimeric G proteins are membrane-associated proteins required for receptor signaling in eukaryotic cells. In response to binding of the appropriate ligand, the receptor stimulates the exchange of bound GDP for GTP on the α subunit, resulting in the dissociation of the α subunit from the β and γ subunits. The GTP-bound α subunit has been shown to directly regulate the activity of downstream effectors (Gilman, A. G. (1987) Ann. Rev. Biochem. 56:615-649; Simon et al. (1991) Science 252:802-808; Bimbaumer, L. (1992) Cell 71:1069-1072). Gilman demonstrated that after dissociating from the GTP-bound α subunit, the βγ subunit exists as a tightly associated complex in vivo. This complex has been found to regulate the activity of a specific subset of downstream effectors, including adenylyl cyclase subtypes II and IV, phospholipase A2, phospholipase C subtypes β1, 2, 3, and various K⁺ and Ca²⁺ ion channels (Tang, W. J. and Gilman, A. G. (1991) Science 254:1500-1503; Wickman et al. (1994) Nature 368:254-257; Clapham, D. E. and Neer, E. J. (1993) Nature 365:403-406). Thus, the G protein α and βγ subunits produce bifurcating signals that regulate the function of these effectors. Moreover, the βγ subunits can directly bind to a receptor (Phillips, W. J. and Cerione, R. A. (1992) J. Biol. Chem. 24:1703-17039) and can increase agonist-dependent phosphorylation and desensitization by directly interacting and recruiting the β-adrenergic (β-ARK) kinases to the membrane (Haga, K. and T. Haga (1992) J. Biol. Chem. (1992) 267:2222-2227; Pitcher et al. (1992) Science 257:1264-1267). Thus, the βγ subunits are important for both effector regulation and receptor recognition.

Multiple isoforms of each of the α, β, and γ subunits of the G proteins has been found in a wide variety of eukaryotic species. Complete cDNAs encoding five distinct mammalian β subunits (β₁-γ₅) have been identified thus far (Watson et al. (1994) J. Biol. Chem. 269:22150-22156). A rat heart cDNA recently identified may encode a sixth β subunit, which is 96% identical to the human β₃ subunit (Ray, K. and Robishaw, J. D. (1994) Gene 149:337-340). At the amino acid level, the β subunits are highly conserved.

In contrast, the γ subunits are much more divergent. In humans and mice, there are at least 16 α, 5 β, and 12γ subunits, which have the potential to form 960 different combinations (Cheng et al. (2003) Developmental Dynamics 228:555-567). The large number of α, β, and γ subunits has raised fundamental questions regarding how the G proteins are assembled to control the specificity of the several hundred receptor signaling pathways that are known to exist. It is not known whether all possible combinations exist in vivo, or what specialized role the various combinations might have (Cheng et al. (2003) Developmental Dynamics 228:555-567). Thus, it is believed that the γ subunit determines the functional specificity of the βγ subunit complex. Different cDNAs of γ subunits have been isolated, for example, the γ₁, subunit from bovine retina (Hurley et al. (1984) Proc. Natl. Acad. Sci USA 81:6948-6952), the γ₂, γ₃, and γ₇ subunits from bovine brain (Robishaw et al. (1989) J. Biol. Chem. 264:15758-15761; Gautam et al. (1989) Science 244:971-974; Gautam et al. (1990) Proc. Natl. Acad. Sci. USA (1990) 87:7973-7977; Cali et al. (1992) J. Biol. Chem. 267:24023-24027), and the γ₅ subunit from bovine and rat liver (Fisher, K. and N. N. Aronson, (1992) Mol. Cell Biol. 12:1585-1591). The existence of a putative γ₄ subunit has also been reported with the isolation of a PCR fragment from mouse kidney and retina (Gautam et al. (1990) Proc. Natl. Acad. Sci. USA 87:7973-7977). In addition, Robishaw and Kunsch reported the cloning and characterization of the human γ₂, γ₃, γ₄, γ₅, γ₇, γ₁₀ and γ₁₁ subunits (WO 96/37513).

The large number of G protein subunit combinations presents a challenge for cost and labor intensive genetic manipulation-based analysis in mice. However, the zebrafish (Danio rerio), produces large a number of eggs, has a short generation time and is easily accessible eggs for genetic manipulation, thus facilitating such analysis. This application demonstrates at the first time a heterotrimeric G protein γ₂ subunit (GNG2) has an essential function in angiogenesis, thus provides the fundamental basis for the potential application of relevant polynucleotides, polypeptides, antibodies and chemicals against GNG2 for modulating angiogenesis in animals.

### SUMMARY OF THE INVENTION

The invention provides isolated nucleic acid sequences encoding the zebrafish GNG2 protein. In some embodiments, the GNG2 encoding nucleic acids have the polynucleotide sequence of SEQ ID NO: 1. The invention also provides GNG-encoding polynucleotides and portions, homologs and fragments thereof from other vertebrate species for use in the methods of the invention. In some embodiments, the polynucleotides are derived from such species as human, mouse, rat, and cow, for example. In some embodiments, the GNG2-encoding polynucleotides have a nucleic acid sequence of SEQ ID NO: 7, SEQ ID NO: 11, SEQ ID NO: 15, SEQ ID NO: 56, SEQ ID NO: 58, SEQ ID NO: 59 or SEQ ID NO: 60.

The invention also provides isolated zebrafish GNG2 polypeptides. In some embodiments, the polypeptide has the amino acid sequence of SEQ ID NO: 2. The invention also provides GNG polypeptides and portions, homologs and fragments thereof from other vertebrate species for use in the methods of the invention. In some embodiments, the polypeptides are derived from such species as human, mouse, cow and rat, for example. In some embodiments, the GNG2 polypeptides have an amino acid sequence of SEQ ID NO: 3, SEQ ID NO: 4, SEQ ID NO: 5, SEQ ID NO: 6, or SEQ ID NO: 57.

The invention also provides methods of promoting angiogenesis in an animal. In some embodiments, the method comprises administering to the animal an effective amount of a GNG2 polypeptide. The polypeptide may be conjugated with various effective delivery reagents to facilitate the passage of the cell membrane barrier. The GNG2 polypeptide may be any vertebrate GNG2 polypeptide (i.e., derived from any species). In some embodiments the GNG2 polypeptide comprises a consensus amino acid sequence of SEQ ID NO: 6. In some embodiments the GNG2 polypeptide comprises the amino acid sequence of SEQ ID NO: 2. In other embodiments the GNG2 polypeptide comprises the amino acid sequence of SEQ ID NO: 4. In other embodiments the GNG2 polypeptide comprises the amino acid sequence of SEQ ID NO: 5.

In other embodiments of the invention, methods for promoting angiogenesis include administering to an animal in need of angiogenesis an effective amount of a polynucleotide encoding a GNG2 polypeptide alone or in combination with any G protein β subunit polynucleotide/polypeptide. The polynucleotide may encode any GNG2 polypeptide. In some embodiments the polynucleotide comprises the nucleic acid sequence of SEQ ID NO: 1. In other embodiments the polynucleotide comprises the nucleic acid sequence of SEQ ID NO: 7. In other embodiments the polynucleotide comprises the nucleic acid sequence of SEQ ID NO: 11. In other embodiments the polynucleotide comprises the nucleic acid sequence of SEQ ID NO: 12. In other embodiments the polynucleotide comprises the nucleic acid sequence of SEQ ID NO: 15. In other embodiments the polynucleotide comprises the nucleic acid sequence of SEQ ID NO: 56. In other embodiments the polynucleotide comprises the nucleic acid sequence of SEQ ID NO: 58. In other embodiments the polynucleotide comprises the nucleic acid sequence of SEQ ID NO: 59. In other embodiments the polynucleotide comprises the nucleic acid sequence of SEQ ID NO: 60.

The invention also provides a method of promoting angiogenesis in an animal in need thereof, comprising administering to an animal an effective amount of a first polynucleotide encoding a GNG2 polypeptide and an effective amount of a second polynucleotide encoding a VEGF polypeptide. In some embodiments, the GNG2 polypeptide comprises an amino acid sequence selected from the group consisting of SEQ ID NO: 2, SEQ ID NO: 4, SEQ ID NO: 5 and SEQ ID NO: 6. In some embodiments, the VEGF polypeptide comprises an amino acid sequence selected from the group consisting of SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, and SEQ ID NO: 52.

The invention also provides methods of inhibiting angiogenesis in an animal. In some embodiments, the method comprises administering to the animal an effective amount of a polynucleotide, such as antisense oligonucleotide, that inhibits the expression of a GNG2 polypeptide. The GNG2 polypeptide may be any vertebrate GNG2 polypeptide (including human, mouse, cow, rat, zebrafish and all other vertebrates). In some embodiments the GNG2 polypeptide comprises a consensus amino acid sequence of SEQ ID NO: 6. In some embodiments the GNG2 polypeptide comprises the amino acid sequence of SEQ ID NO: 2. In other embodiments the GNG2 polypeptide comprises the amino acid sequence of SEQ ID NO: 4. In other embodiments the GNG2 polypeptide comprises the amino acid sequence of SEQ ID NO: 5. In other embodiments the polynucleotide comprises the nucleic acid sequence of SEQ ID NO: 3. In other embodiments the polynucleotide comprises the nucleic acid sequence of SEQ ID NO: 6. In other embodiments the polynucleotide comprises the nucleic acid sequence of SEQ ID NO: 57.

The invention also provides a method of treating an angiogenesis-related disease comprising administering to a patient in need of such treatment a polynucleotide that inhibits the expression of GNG2 in an amount sufficient to inhibit angiogenesis, or in an amount that would enhance the efficacy of any combined regimen of therapy, such as anti-VEGF and chemotherapy. An angiogenesis-related disease includes, but is not limited to angiogenesis-dependent cancers; benign tumors; rheumatoid arthritis; psoriasis; ocular angiogenesis diseases; Osler-Webber Syndrome; myocardial angiogenesis; plaque neovascularization; telangiectasia; hemophiliac joints; angiofibroma; wound granulation; intestinal adhesions, atherosclerosis, scleroderma, hypertrophic scars, cat scratch disease and Helicobacter pylori ulcers.

As such, the method encompasses treating a patient with an angiogenesis-dependent tumor by administering a polynucleotide, modified polynucleotide or in combination with vehicles that allow effective delivery of polynucleotides into the cells and tissues, and that inhibit the expression of GNG2 in an amount sufficient to cause tumor regression or stabilization of the cancer condition, or in an amount that would enhance the efficacy of any combined regimen of therapy. The polynucleotide or modified polynucleotide may be an antisense oligonucleotide, siRNA, a morpholino oligonucleotide, or a ribozyme that specifically hybridizes to a nucleic acid encoding GNG2. The invention also provides a method for treating a subject with cell-permeable peptides to inhibit the interaction of GNG2 with other α or β subunits, upstream receptors, or downstream effectors. The methods of the invention may further include cell-permeable peptide inhibition of VEGF activity.

In some embodiments, the invention provides a method for treating an angiogenesis-related disease comprising administering to a patient in need of such treatment a first reagent that inhibits the expression of GNG2 and a second reagent that inhibits the expression of VEGF, wherein the first reagent and the second reagent together act synergistically to inhibit angiogenesis. The first reagent may comprise a polynucleotide (including antisense molecules, such as RNAis, siRNAs, mRNAs, modified nucleic acids, PNAs, or morpholino oligonucleotides or ribozymes, etc.) directed against a GNG2-encoding polynucleotide. In some embodiments, the first reagent has a sequence selected from the group consisting of SEQ ID NO: 8, SEQ ID NO: 9, SEQ ID NO: 10, SEQ ID NO: 12 and combinations thereof. It is not as straight forward to define only a small region of the targeted sequence for antisense morpholino or other siRNA. Translational blocking antisense morpholino oligos work anywhere from 5′ untranslated region to 25 bp downstream of the ATG start codon. In addition, splicing inhibiting antisense morpholino oligos work between exon/intron boundaries. Thus, any exon/intron boundaries of the GNG2 gene are target sequences. Therefore, any region of the GNG2 mRNA transcript for translational blocking antisense and any genomic region of the GNG2 gene for splicing inhibition may be used. The human GNG2 mRNA accession number is NM_{—}053064, Unigene Cluster Hs.112928 (Ensembl Gene ID: ENSG00000186469 from the Sanger Institute). The second reagent may comprise a polynucleotide (including antisense molecules, RNAis, siRNAs, mRNAs, modified nucleic acids, PNAs, or morpholino oligonucleotides or ribozymes, etc.) directed against a VEGF-encoding polynucleotide. In some embodiments, the first reagent has a sequence selected from the group consisting of SEQ ID NO: 53, SEQ ID NO: 54, SEQ ID NO: 55 and combinations thereof. The human VEGF accession number is NM_{—}003376, Unigene cluster: Hs.73793 (Ensembl gene ID: ENSG00000112715). In some embodiments, the angiogenesis-related disease is related to tumor or cancer progression.

In order to design oligonucleotides to disrupt the function of GNG2 and/or VEGF, one may use the various computer-based algorithms as are known in the art for predicting sequences that are likely to have the desired affect of inhibiting RNA expression. An example of such an algorithm includes, but is not limited to Sfold as is available on the Sfold webserver on the world wide web under the domain address of sfold.wadsworth,org. This algorithm is also described in Ding, Y. et al. (2004) Nucl. Acids Res. 32:W135-W141.

In some embodiments, the first reagent is a small molecule, a natural compound or a synthetic compound from combinatorial chemistry that inhibits the expression or function of GNG2 or VEGF. Such compounds may be those known in the art or may be identified using the screening methods described herein. Such compounds may, for example, inhibit the expression of GNG2, the post-translation al modification of GNG2 (e.g., affect prenylation), alter the allosteric conformation of GNG2, and/or affect the interaction of the βγ dimer.

The invention further provides a method of treating an angiogenesis-related disease comprising administering to a patient in need of such treatment a first compound that inhibits the function or expression of GNG2 and a second compound that inhibits the expression or function of VEGF signaling, including its receptors and downstream signaling molecules, wherein said first compound and said second compound are provided in sufficient amounts to inhibit angiogenesis. In some embodiments, the compound that inhibits the function of VEGF is an antibody that specifically binds VEGF.

In a particular embodiments of the invention, the invention provides a method of treating a patient with an angiogenesis-dependent tumor comprising administering to a patient in need of such treatment a first compound that inhibits the expression or function of GNG2 and a second compound, (including a natural compound, a synthetic compound or a small molecule from combinatorial chemistry), that inhibits the expression or function of VEGF, wherein said first compound and said second compound are provided in sufficient amounts to cause tumor regression. In some embodiments, the compound that inhibits the expression or function of GNG2 is a polynucleotide, such as an antisense, morpholino oligonucleotide, or ribozyme that specifically hybridizes to a nucleic acid comprising a sequence that encodes a GNG2 polypeptide. In other embodiments, the compound that inhibits the expression or function of GNG2 is an antibody that specifically binds a GNG2 polypeptide. In some embodiments, the compound that inhibits the expression or function of VEGF is a polynucleotide, such as an antisense, morpholino oligonucleotide, or ribozyme that specifically hybridizes to a nucleic acid comprising a sequence that encodes a VEGF polypeptide. In other embodiments, the compound that inhibits the function of VEGF is an antibody that specifically binds a VEGF polypeptide. In some embodiments the compounds that inhibit GNG2 and/or VEGF expression and/or function are combinations of polynucleotides and antibodies.

### BRIEF DESCRIPTION OF THE DRAWINGS

FIG. 1 shows an alignment of the nucleotide sequence of zebrafish GNG2 (SEQ D NO: 1) and the encoded amino acid sequence (SEQ ID NO: 2).

FIG. 2 shows an alignment of the amino acid sequences of human GNG2 (SEQ ID NO: 4) with the zebrafish GNG2 (SEQ ID NO: 2) and the consensus sequence (SEQ ID NO: 5). A blank space indicates a non-conserved amino acid difference. A “+” indicates a conserved amino acid difference.

FIG. 3 shows an alignment of the amino acid sequences of human GNG2 (SEQ ID NO: 4) with the mouse GNG2 (SEQ ID NO: 5) and the zebrafish GNG2 (SEQ ID NO: 2). A blank space indicates a non-conserved amino acid difference. An asterisk indicates a conserved amino acid difference. Sequence and expression analysis of gng2. (A) Zebrafish Gγ2 protein is 94% identical to mammalian proteins.

FIG. 4 shows an alignment of members of the human GNG family graphically showing conservation of amino acid residues. The sequences are: AAH29367 (GNGT1) (SEQ ID NO: 18); AAM12592 (GNG11) (SEQ ID NO: 19); AAB70039 (GNGT2) (SEQ ID NO: 20); AAM12590 (GNG9) (SEQ ID NO: 21); AAM12585 (GNG3) (SEQ ID NO: 22); AAM12586 (GNG4) (SEQ ID NO: 23); AAF04569 (GNG8) (SEQ ID NO: 24); AAM12588 (GNG7) (SEQ ID NO: 25); AAF04571 (GNG12) (SEQ ID NO: 26); AAM12587 (GNG5) (SEQ ID NO: 27); AAF04568 (GNG5L) (SEQ ID NO: 28); AAM12591 (GNG10) (SEQ ID NO: 29); AAM12594 (GNG13) (SEQ ID NO: 30).

FIG. 5 shows a phylogram of the human GNG family. The sequences are: AAH29367 (GNGT1) (SEQ ID N018); AAM12592 (GNG11) (SEQ ID NO: 19); AAB70039 (GNGT2) (SEQ ID NO: 20); AAM12590 (GNG9) (SEQ ID NO: 21); AAM12585 (GNG3) (SEQ ID NO: 22); AAM12586 (GNG4) (SEQ ID NO: 23); AAF04569 (GNG8) (SEQ ID NO: 24); AAM12588 (GNG7) (SEQ ID NO: 25); AAF04571 (GNG12) (SEQ ID NO: 26); AAM12587 (GNG5) (SEQ ID NO: 27); AAF04568 (GNG5L) (SEQ ID NO: 28); AAM12591 (GNG10) (SEQ ID NO: 29); AAM12594 (GNG13) (SEQ ID NO: 30).

FIG. 6 (A) shows a phylogram analysis that reveals that zebrafish Gγ2 protein is highly conserved among vertebrates. The Gγ2 protein contains a CAA (L/S) box (rectangular box, where A was any aliphatic amino acid) at the C-terminus which is conserved among the Gγ2 protein family. (B) shows RT-PCR detection of the maternal gng2 transcript from 1-cell to 128-cell stage and zygotic transcript from high stage onward to 1 dpf.

FIG. 7 shows GNG2 expression in neural tube and axial vasculature tissues at 24 hours post-fertilization (hpf). Panel A is a lateral view; Panel B is a dorsal view (inset: magnified). Panels A and B show gng2 expression in the neural tube and axial vascular tissues at 1 dpf, and Panel C shows the otic vesicles in 2 dpf in zebrafish embryos as detected by whole-mount in situ hybridization. (v.m.h. ventral mid- and hind-brain; tel. telecephalon; a.v. axial vasculature; s.n. spinal cord neurons; o.v. otic vesicles; f.b. forebrain; h.b. hindbrain; p.p. pharyngeal pouch; o.t. outflow tract; s.v. sprouting vessels). All scale bars are 100 μm.

FIG. 8 shows the translational inhibition morpholino against GNG2. MO-gng2 (translation) (SEQ ID NO: 8) is the reverse complement of GNG2 (sense) (SEQ ID NO: 9). The complement of SEQ ID NO: 9 (MO-gng2 (antisense) is the same as SEQ ID NO: 8 shown in the 3′ to 5′ direction. The MO-gng2 (translation) (SEQ ID NO: 8) is shown within the context of the zebrafish GNG2 gene (SEQ ID NO: 11) as boldfaced and underlined.

FIG. 9 shows the splicing morpholino against GNG2 mRNA splicing. MO-GNG2 (splicing) (SEQ ID NO: 12) is also shown within the context of the zebrafish GNG2 gene exon-intron-exon (SEQ ID NO: 13). The exons are shown double underlined and the MO-GNG2 (splicing) (SEQ ID NO: 12) is shown in boldface capital letters.

FIG. 10 shows targeted suppression of GNG2 which results in a shortening of the zebrafish body axis, as well as mesodermal and neural abnormalities. Panel A shows a wild type embryo; Panel B shows an embryo following treatment with an MO-GNG2 translation morpholino; and Panel C shows an embryo following treatment with an MO-GNG2 splicing morpholino.

FIG. 11 shows targeted knockdown of gng2 using splicing morpholino in zebrafish embryos. (A) Splice junction morpholino targeted against gng2 exon-intron boundary. (B) RT-PCR of gng2 transcript at tailbud stage in wildtype (WT) and gng2-MO (100 μM) morpholino injected embryos, comparing cryptic spliced transcript in the morpholino injected embryos to the cDNA and genomic PCR products.

FIG. 12 shows the wild type GNG2 mRNA and splice variant mRNA resulting from MO-GNG2 directed missplicing of zebrafish RNA. The nucleotide sequences of wild type GNG2 (SEQ ID NO: 15) and misspliced GNG2 (SEQ ID NO: 16) are shown with the respective deduced amino acid sequences (wild type, SEQ ID NO: 2; splice variant, SEQ ID NO: 17).

FIG. 13 shows essential function of G protein γ₂ for angiogenesis in vivo. (A) and (D) flk1 expression in WT zebrafish embryos at 1 dpf, in axial vasculature and inter-somitic vessels (IS) (D, arrow) by whole-mount in situ hybridization. (B-C) and (E-F) gng2-splicing knockdown (gng2-MO, 100 μM) inhibited the formation of inter-somitic vessel, a process of angiogenesis, (B) and (E) were complete loss of IS, 53% of embryos, (C) and (F) were partial loss of IS, 43% embryos, (A-F) were results from N=110, 5 experiments). (G-H) Targeting both G protein (gng2-MO, 40 μM) and vegf (vegf-MO, 25 μM) using antisense morpholinos dramatically increased the efficacy of anti-angiogenesis as visualized by loss of sprouting of inter-somitic vessels from the dorsal aorta at 1 dpf zebrafish embryos (H). vegf knockdown alone (vegf-MO, 25 μM) at the sub-effective dose showed no effect (G). Inserts in (G-H) are higher magnification of IS, (G,H arrows). Injection volume was about 1 nl. All scale bars are 100 μm (A-H).

FIG. 14 shows VEGF-mRNA overexpression induced endothelial expression of flk at 1 dpf. Panel A shows wild type, 1 dpf, (lateral view); Panel B shows VEGF-mRNA, 1 dpf, (lateral view); Panel C shows wild type, 1 dpf, (lateral view, magnified); Panel D shows VEGF-mRNA, 1 dpf, (lateral view, magnified); Panel E shows wild type, 1 dpf, (dorsal view); and Panel F shows VEGF-mRNA, 1 dpf, (dorsal view).

FIG. 15 shows G protein γ₂ modulated VEGF signaling pathway in vivo. (A-C) vegf mRNA (20 ng/μl) overexpression can increase level of flk1 transcripts in the axial vasculature and the inter-somitic vessels in zebrafish embryos (compare to WT control in FIGS. 13A and 13D). (D-F) vegf mRNA overexpression followed by targeted knockdown of G protein (gng2-MO, 100 μM) specifically inhibited sprouting of inter-somitic vessels from the dorsal aorta (D-E), and strikingly, the yolk common cardinal vein was completely inhibited (F, arrow). (G-H) vegf mRNA over-expression can activate PLCγ1 as detected by anti-phospho PLCγ1 antibody (H, arrow) compared to WT control (G). (I) Knockdown of gng2 using gng2-MO splicing morpholino specifically inhibited vegf activation of PLCγ1 (I, arrow). (J-K) vegf mRNA overexpression can also activate AKT as detected by anti-phospho AKT antibody (K, arrow) compared to WT control (J). (L) Knockdown of gng2 specifically inhibited vegf activation of AKT (L, arrow). vegf mRNA (20 ng/μl) and gng2-MO splicing morpholino (100 μM) were injected at about 1 nl volume at 1-cell stage embryo and followed by whole-mount immunohistochemical staining at 1 dpf. WT control (G) and (J), vegf mRNA overexpression (A-C, H-K), and co-injection of vegf mRNA and gng2-MO (D-F, I-L). All scale bars are 100 μm.

FIG. 16 shows that suppression of GNG2 (MO-GNG2) enhances the anti-angiogenic effect of blocking VEGF signaling (MO-VEGF) as revealed by flk expression. Panel A shows MO-GNG2 (40 μM)+MO-VEGF (25 μM), 1 dpf, (lateral view); Panel B shows MO-GNG2 (40 μM)+MO-VEGF (25 μM), 1 dpf, (lateral view, magnified); Panel C shows MO-VEGF (25 μM), 1 dpf, (lateral view); Panel D shows MO-VEGF (25 μM), 1 dpf, (lateral view, magnified); Panel E shows wild type, 1 dpf, (lateral view); and Panel F shows MO-GNG2 (40 μM), 1 dpf, (lateral view).

FIG. 17 shows a model of essential function of G protein in VEGF signaling during angiogenesis. It is hypothesized that the heterotrimeric G-protein, composed of α, β and γ2 subunits, is essential for the activation of PLCγ1 and AKT in the VEGF signaling pathway.

FIG. 18 shows translational blocking by gng2-ATG morpholino detected by in vitro cell lysate assay. (A) Translational blocking morpholino, targeted against the 5′UTR spanning the ATG start codon of gng2 open reading frame, fused to V5-6× Histidine epitope. (B) Dose dependent inhibition of gng2 translation by gng2-ATG morpholino using in vitro cell lysate. No inhibition by 5-base mismatch control morpholino. Anti-V5 antibody was used to detect the in vitro translation of gng2-V5 fusion protein. (C) WT control zebrafish embryo at 1 dpf. (D) gng2-ATG-MO knockdown embryo at 1 dpf. All scale bars are 100 μm (C-D).

FIG. 19 shows stills from videos of blood flow in zebrafish embryos. Loss-of-function of gng2 led to blood flow defect at the inter-somitic vessels in developing zebrafish embryos. Blood circulation in the vasculatures was monitored as a functional assay of vessel development in zebrafish embryos. Various morpholinos against gng2 were microinjected into 1-cell stage zebrafish embryos. The first morphological sign of angiogenic defect was detected at about 3 dpf when the knockdown embryos showed reduced or missing blood flow to the intersomitic vessels, although there was blood circulation in the dorsal aorta and cardinal vein. The inter-somitic vessels are particularly interesting since they develop by sprouting from the dorsal aorta and cardinal vein, a process that resembles angiogenesis in physiological and pathophysiological processes. Movie S1-S4 are enlarged trunk regions. All scale bars are 100 μm. Panel A shows blood circulation in WT control zebrafish embryo at 3 dpf (wherein the blood flows in the inter-somitic vessels). Panel B shows blood flow in gng2-MO splicing knockdown embryo at 3 dpf (showing blood flow in the dorsal aorta and cardinal vein but the flow to the inter-somitic vessels was blocked or reduced); Panel C shows blood flow of gng2-ATG-MO translational blocking morpholino knockdown embryo at 3 dpf (showing blood flow in the dorsal aorta and cardinal vein but the flow to the inter-somitic vessels is blocked or reduced); Panel D shows blood flow in 5-base mismatch control morpholino (5-mismatch-MO) knockdown embryo at 3 dpf (showing normal blood flow in the inter-somitic vessels).

FIG. 20 shows an in situ hybridization of a mouse embryo showing expression of mouse gng2 in forebrain, midbrain, hindbrain, spinal cord and developing vasculature at E9.5 stage.

### DETAILED DESCRIPTION OF ILLUSTRATIVE EMBODIMENTS

The roles of the G protein βγ dimers in regulating the activity of a specific subset of downstream effectors including adenylyl cyclase subtypes II and IV, phospholipase A2, phospholipase C subtypes β₁, β₂, β₃, and K⁺ and Ca²⁺ channels, and receptor recognition has increased the importance of identifying and characterizing these proteins. Functional specificity of these dimers is believed to be determined by the γ subunit. Striking differences between the retinal and brain βγ subunits have been reported in terms of membrane association (Lee et al. (1992) J. Biol. Chem. 267:24776-24781), interaction with G protein α subunits, receptors (Fawzi et al. (1991) J. Biol. Chem. 266:12194-12200), receptor kinases (Pitcher et al. (1992) Science 257:1264-1267), and effectors (Iniguez-Lluhi et al. (1992) J. Biol. Chem. 32:23409-23410). Since the retinal and brain βγ subunits share a common β₁ subunit, these differences appear to be due to their unique γ subunit.

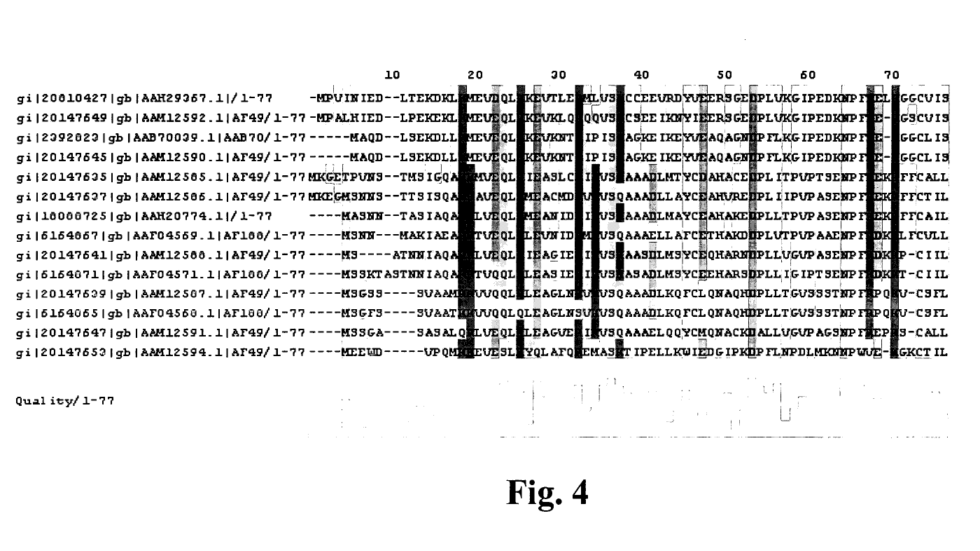

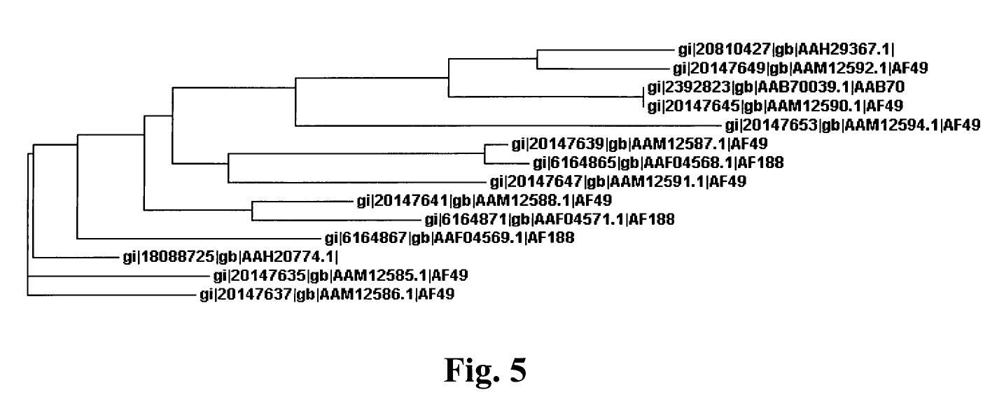

Analysis of the amino acid sequence conservation of γ subunits suggests that the subunit family can be divided into four distinct subclasses, one containing γ₁, γ₁₁, γ_{T1}, γ_{T2} and γ₁₄ subunits, a second containing the γ₇ and γ₁₂ subunits, a third containing the γ₅ and γ₁₀ subunits, and a fourth containing the γ₁₃ subunit Robishaw, J. D., Schwindinger, W. F and Hansen, C. A. (2003) Specificity of G protein βγ dimer signaling. Elsevier Science, USA. HANDBOOK OF CELL SIGNALING, v. 2. pp 623-629) (See FIGS. 4 and 5). These subclasses are based not only on amino acid homology, but also on functional similarities. Thus, within a subclass, members display similar post-translational modifications and similar abilities to interact with the β and α subunits of the G proteins. For example, it appears that the γ₂ subunit is modified by a geranylgeranyl group, interacts with the β₂ subunit, and interacts at least to some extent, with the α₀ subunit.

The present invention provides polynucleotides and polypeptides for the zebrafish γ₂ subunit (GNG2) as well as methods of promoting and inhibiting angiogenesis in animal using GNG2 sense and antisense polynucleotides, and polypeptides derived from any vertebrate species, particularly human, mouse, rat, cow and zebrafish.

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Various definitions are made throughout this document. Most words have the meaning that would be attributed to those words by one skilled in the art. Words specifically defined either below or elsewhere in this document have the meaning provided in the context of the present invention as a whole and as are typically understood by those skilled in the art. Any conflict between an art-understood definition of a word or phrase and a definition of the word or phrase as specifically taught in this specification shall be resolved in favor of the latter. Headings used herein are for convenience and are not to be construed as limiting.

Standard reference works setting forth the general principles of recombinant DNA technology known to those of skill in the art include Ausubel et al., CURRENT PROTOCOLS IN MOLECULAR BIOLOGY, John Wiley & Sons, New York, 1998; Sambrook et al., MOLECULAR CLONING: A LABORATORY MANUAL, 2D ED., Cold Spring Harbor Laboratory Press, Plainview, N.Y., 1989; Kaufman et al., Eds., HANDBOOK OF MOLECULAR AND CELLULAR METHODS IN BIOLOGY AND MEDICINE, CRC Press, Boca Raton, 1995; McPherson, Ed., DIRECTED MUTAGENESIS: A PRACTICAL APPROACH, IRL Press, Oxford, 1991. Standard works setting forth general principles and protocols for working with zebrafish known to those of skill in the art include, but are not limited to THE ZEBRAFISH BOOK: A GUIDE FOR THE LABORATORY USE OF ZEBRAFISH (DANIO RERIO), Westerfield, M., 4th ed., Univ. of Oregon Press, Eugene, 2000.

As used herein “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

A “purified” or “substantially purified” polynucleotide or polypeptide is substantially separated from other cellular components that naturally accompany a native (or wild-type) nucleic acid or polypeptide and/or from other impurities (e.g., agarose gel). A purified polypeptide or protein will comprise about 60% to more than 99% w/w of a sample, and may be about 90%, about 95%, or about 98% pure.

“About” as used herein refers to +/−10% of the reference value.

As used herein, “variant” nucleotide or amino acid sequences refer to homologs, including, for example, isoforms, species variants, allelic variants, and fragments of the sequence of interest. “Homologous nucleotide sequence” or “homologous amino acid sequence,” or variations thereof, refers to sequences characterized by a percentage identity (if referring to polynucleotides) or homology (if referring to polypeptides) of at least about 60%, at least about 70%, at least about 75%, at least about 80%, at least about 81%, at least about 82%, at least about 83%, at least about 84%, at least about 85%, preferably at least about 90%, at least about 95%, at least about 98%, or at least about 99%, and more preferably 100%, with respect to a reference sequence, or portion or fragment thereof encoding or having a functional domain.

The term “therapeutically effective amount” of a GNG2 antagonist, such as a GNG2 antisense oligonucleotide, means an amount calculated to achieve and maintain a therapeutically effective level in the disease state (e.g., a tumor, if applied to a tumor), or in the plasma, if administered systematically, so as to inhibit angiogenesis (and, if applied to cancer, to inhibit the proliferation of cancer cells). By way of example, the therapeutic amount sufficient to inhibit proliferation of more than about 50 percent of cancer cells, such as KS cells, in vitro. Of course, the therapeutic dose will vary with the potency of each GNG2 antagonist in inhibiting cancer cell growth in vitro, and the rate of elimination or metabolism of the GNG2 antagonist by the body in the tumor tissue and/or in the plasma. The therapeutic dose also applies to an amount of GNG2 antagonist that would enhance the efficacy of any combined regimen of therapy, such as anti-VEGF and chemotherapy.

1. Polynucleotides

A. Vertebrate GNG2

The invention provides polynucleotides encoding the zebrafish GNG2 polypeptide and also provides polynucleotides encoding GNG2 derived from other vertebrates for use in the methods of the invention. As used herein “polynucleotide” refers to a nucleic acid molecule and includes genomic DNA, cDNA, RNA, mRNA, mixed polymers, recombinant nucleic acids, fragments and variants thereof, and the like. Polynucleotide fragments useful in the invention comprise at least 10, and preferably at least 12, 14, 16, 18, 20, 25, 30, 35, 40, 45, 50, 75, or 100 consecutive nucleotides of a reference polynucleotide. The polynucleotides of the invention include sense and antisense strands. The polynucleotides of the invention may be naturally occurring or non-naturally occurring polynucleotides. A “synthesized polynucleotide” as used herein refers to polynucleotides produced by purely chemical, as opposed to enzymatic, methods. “Wholly” synthesized DNA sequences are therefore produced entirely by chemical means, and “partially” synthesized DNAs embrace those wherein only portions of the resulting DNA were produced by chemical means. The polynucleotides of the invention may be single- or double-stranded. The polynucleotides of the invention may be chemically modified and may contain non-natural or derivatized nucleotide bases as will be readily appreciated by those skilled in the art. Such modifications include, for example, labels, methylation, substitution of one or more nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides, etc.), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

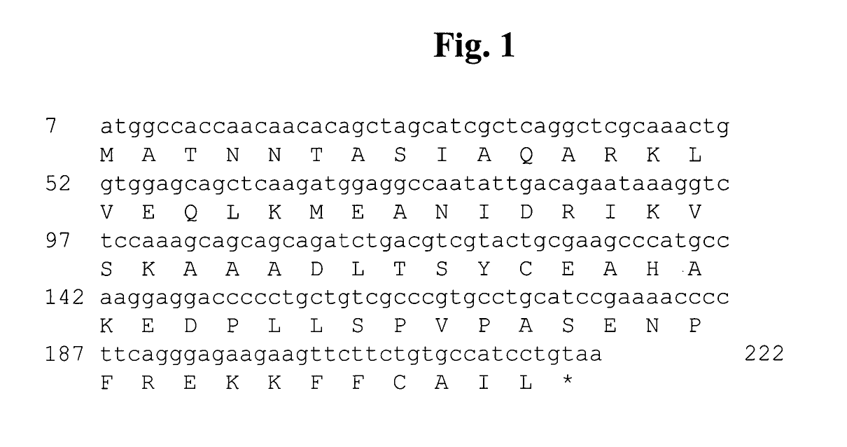

The polynucleotides of the invention include those that encode the polypeptide sequence of SEQ ID NO: 2 (see for example FIG. 1). In some embodiments, the polynucleotides comprise the nucleic acid sequence of SEQ ID NO: 1. The polynucleotides may contain mutations that result in amino acid changes that are either conservative or non-conservative. Mutations can be introduced into a nucleic acid sequence of the invention by standard techniques, such as site-directed mutagenesis and PCR-mediated mutagenesis. Conservative amino acid substitutions may be made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (for example, lysine, arginine, and histidine), acidic side chains (for example, aspartic acid, glutamic acid), uncharged polar side chains (for example, glycine, asparagine, glutamine, serine, threonine, tyrosine, cysteine), nonpolar side chains (for example, alanine, valine, leucine, isoleucine, proline, phenylalanine, methionine, tryptophan), beta-branched side chains (for example, threonine, valine, isoleucine), and aromatic side chains (for example, tyrosine, phenylalanine, tryptophan, histidine). Thus, a predicted nonessential amino acid residue is replaced with another amino acid residue from the same side chain family. Alternatively, mutations can be introduced randomly along all or part of a coding sequence, such as by saturation mutagenesis, and the resultant mutants can be screened for biological activity to identify mutants that retain activity. Following mutagenesis, the encoded protein may be expressed by any recombinant technology known in the art and the activity of the protein can be determined.

The nucleotide sequences are presented by single strands only, in the 5′ to 3′ direction, from left to right. Nucleotides are represented in the manner recommended by the IUPAC-IUB Biochemical Nomenclature Commission.

In some embodiments of the invention the mutations will not significantly alter the post-translational modifications or biological activity of the protein. For example, the GNG2 polypeptides should retain the ability to be modified by geranylgeranyl groups, and interact with β subunits, and at least to some extent, with α subunits of G proteins.

B. Expression Vectors

Another aspect of the invention pertains to vectors, preferably expression vectors, containing a nucleic acid encoding zebrafish GNG2 polypeptide, or derivatives, fragments, analogs or homologs thereof. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

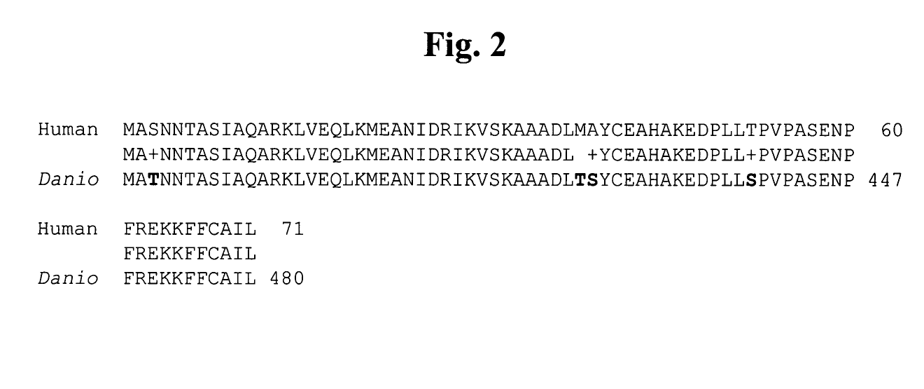

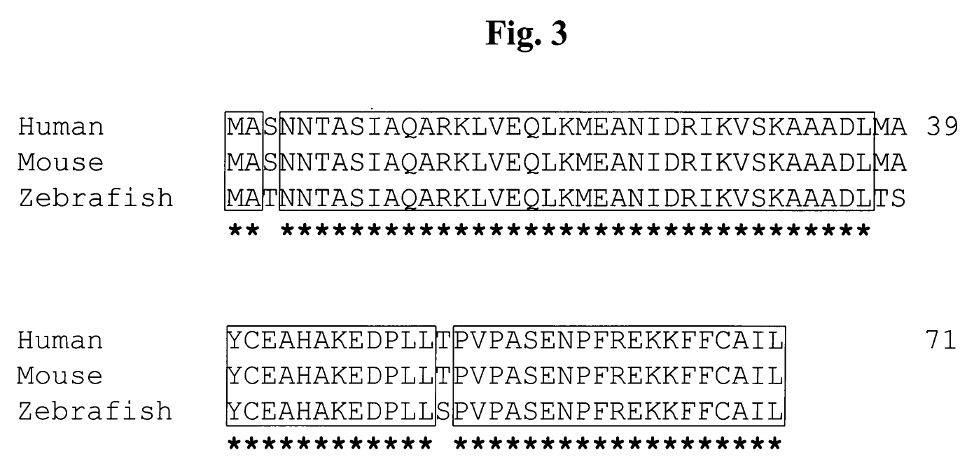

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., GNG2 polypeptides, mutant forms of GNG2, fusion proteins, etc.) from any vertebrate species. In some embodiments, the GNG2 is derived from mammals. In some embodiments, the GNG2 is derived from mice or rats. In other embodiments, the GNG2 is derived from humans. In other embodiments, the GNG2 is derived from zebrafish. A comparison of the amino acid sequences of human and zebrafish gng2 is shown in FIG. 2. An alignment of the amino acid sequences of human, mouse and zebrafish gng2 is shown in FIG. 3.

The recombinant expression vectors of the invention can be designed for expression of vertebrate GNG2 in prokaryotic or eukaryotic cells. For example, zebrafish GNG2 can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc.; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Ammann et al., (1988) Gene 69:301-315) and pET lid (Studier et al., GENE EXPRESSION TECHNOLOGY: METHODS IN ENZYMOLOGY 185, Academic Press, San Diego, Calif. (1990) pp. 60-89).

One strategy to maximize recombinant protein expression in E. coli is to express the protein in a host bacterium with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the vertebrate GNG2 expression vector is a yeast expression vector. Examples of vectors for expression in yeast Saccharomyces cerevisiae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the vertebrate GNG2 can be expressed in insect cells using baculovirus expression vectors. Baculovirus vectors available for expression of proteins in cultured insect cells (e.g., Spodoptera frugiperda SF9 cells) include the pAc series (Smith et al. (1983) Mol. Cell. Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. Representative examples of promoters include, but are not limited to LTR or SV40 promoter, the E. coli, lac or trp, the phage lambda P_{L} promoter and other promoters known to control expression of genes in prokaryotic or eukaryotic cells or their viruses. For other suitable expression systems for both prokaryotic and eukaryotic cells, see, e.g., Chapters 16 and 17 of Sambrook et al., MOLECULAR CLONING: A LABORATORY MANUAL 2nd ed., Cold Spring Harbor Laboratory, Cold Spring Harbor Laboratory Press, Cold Spring Harbor, N.Y., 1989. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, pRITS (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host. In addition, a complete mammalian transcription unit and a selectable marker can be inserted into a prokaryotic plasmid. The resulting vector is then amplified in bacteria before being transfected into cultured mammalian cells. Examples of vectors of this type include pTK2, pHyg and pRSVneo.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev. 3:537-546). Selection of the appropriate vector and promoter is well within the level of ordinary skill in the art.

The expression vector may also contain a ribosome binding site for translation initiation and a transcription terminator. The vector may also include appropriate sequences for amplifying expression. In addition, in some embodiments, the expression vectors contain one or more selectable marker genes to provide a phenotypic trait for selection of transformed host cells such as dihydrofolate reductase (DHFR) or neomycin (NEO) resistance for eukaryotic cell culture, or such as tetracycline (TET) or ampicillin (AMP) resistance in E. coli.

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to vertebrate GNG2 mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., (1986) “Antisense RNA as a molecular tool for genetic analysis,” Reviews—Trends in Genetics, Vol. 1(1):22-25.

C. Antisense Oligonucleotides

Antisense oligonucleotides may be used as therapeutic moieties in the treatment of disease states in animals and man. Antisense oligonucleotide drugs, including ribozymes, have been safely and effectively administered to humans and numerous clinical trials are presently underway. It is thus established that oligonucleotides can be useful therapeutic modalities that can be configured to be useful in treatment regimes for treatment of cells, tissues and animals, especially humans.

In the context of this invention, the term “oligonucleotide” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are contemplated by the present invention, other oligomeric antisense compounds, including but not limited to oligonucleotide mimetics such as are described below, are also included. The antisense compounds in accordance with this invention preferably comprise from about 8 to about 50 nucleobases (i.e. from about 8 to about 50 linked nucleosides). In some embodiments, the antisense oligonucleotides comprise from about 8 to about 15 nucleobases. In other embodiments, the antisense oligonucleotides comprise from about 16 to about 25 nucleobases. In other embodiments, the antisense oligonucleotides comprise from about 20 to about 30 nucleobases. In other embodiments, the antisense oligonucleotides comprise from about 25 to about 35 nucleobases. In other embodiments, the antisense oligonucleotides comprise from about 30 to about 45 nucleobases. In other embodiments, the antisense oligonucleotides comprise from about 40 to about 50 nucleobases. Antisense compounds include ribozymes, external guide sequence (EGS) oligonucleotides (oligozymes), and other short catalytic RNAs or catalytic oligonucleotides which hybridize to the target nucleic acid and modulate its expression.

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

In some embodiments, the modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Preferred oligonucleotides having inverted polarity comprise a single 3′ to 3′ linkage at the 3′-most internucleotide linkage i.e., a single inverted nucleoside residue which may be a basic (the nucleobase is missing or has a hydroxyl group in place thereof). Various salts, mixed salts and free acid forms are also included.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al. (1991) Science 254:1497-1500.

Modified oligonucleotides may also contain one or more substituted sugar moieties.

A further modification includes Locked Nucleic Acids (LNAs) in which the 2′-hydroxyl group is linked to the 3′ or 4′ carbon atom of the sugar ring thereby forming a bicyclic sugar moiety. The linkage is preferably a methylene (—CH₂—)ₙ group bridging the 2′ oxygen atom and the 4′ carbon atom wherein n is 1 or 2. LNAs and preparation thereof are described in WO 98/39352 and WO 99/14226.

Other modifications include 2′-methoxy (2′-O—CH₃), 2′-aminopropoxy (2′-OCH₂CH₂CH₂ NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in THE CONCISE ENCYCLOPEDIA OF POLYMER SCIENCE AND ENGINEERING, pages 858-859, Kroschwitz, J. I., ed. John Wiley & Sons, 1990, those disclosed by Englisch et al. Angewandte Chemie, International Edition, 1991, 30, 613, and those disclosed by Sanghvi, Y. S., Chapter 15, ANTISENSE RESEARCH AND APPLICATIONS, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., ANTISENSE RESEARCH AND APPLICATIONS, CRC Press, Boca Raton, 1993, pp. 276-278) and are presently preferred base substitutions, even more particularly when combined with 2′-O-methoxyethyl sugar modifications.

Another modification of the oligonucleotides of the invention involves chemically linking to the oligonucleotide one or more moieties or conjugates which enhance the activity, cellular distribution or cellular uptake of the oligonucleotide. The compounds of the invention can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Conjugate moieties include but are not limited to lipid moieties such as a cholesterol moiety (Letsinger et al. (1989) Proc. Natl. Acad. Sci. USA 86:6553-6556), cholic acid (Manoharan et al. (1994) Bioorg. Med. Chem. Let. 4:1053-1060), a thioether, e.g., hexyl-S-tritylthiol (Manoharan et al. (1992) Ann. N.Y. Acad. Sci. 660:306-309; Manoharan et al. (1993) Bioorg. Med. Chem. Let. 3:2765-2770), a thiocholesterol (Oberhauser et al. (1992) Nucl. Acids Res. 20:533-538), an aliphatic chain, e.g., dodecandiol or undecyl residues (Saison-Behmoaras et al. (1991) EMBO J. 10:1111-1118; Kabanov et al. (1990) FEBS Lett. 259:327-330; Svinarchuk et al. (1993) Biochimie 75:49-54), a phospholipid, e.g., di-hexadecyl-rac-glycerol or triethyl-ammonium 1,2-di-O-hexadecyl-rac-glycero-3-H-phosphonate (Manoharan et al. (1995) Tetrahedron Lett. 36:3651-3654; Shea et al. (1990) Nucl. Acids Res. 18:3777-3783), a polyamine or a polyethylene glycol chain (Manoharan et al. (1995) Nucleosides & Nucleotides 14:969-973), or adamantane acetic acid (Manoharan et al. (1995) Tetrahedron Lett. 36:3651-3654), a palmityl moiety (Mishra et al. (1995) Biochim. Biophys. Acta 1264:229-237), or an octadecylamine or hexylamino-carbonyl-oxycholesterol moiety (Crooke et al. (1996) J. Pharmacol Exp. Ther. 277:923-937. Oligonucleotides of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic.

It is not necessary for all positions in a given compound to be uniformly modified, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

D. Morpholino-Modified Oligonucleotides

A particular form of antisense technology are morpholino oligonucleotides (Summerton, J. and D. Weller (1997) Antisense Nucl. Acid Drug Dev. 7:187-195; Nasevicius, A. and S. C. Ekker (2000) Nat. Genet. 26:216-220; Yan, Y-K. et al. (2002) Development 129:5065-5079). Morpholino oligonucleotides are nonionic DNA analogs with altered backbone linkages compared with DNA or RNA, but follow Watson-Crick base-pairing with complementary sequences. Typically, morpholinos are at least about 18-25 nucleobases in length, in some embodiments, the morpholinos are at least about 25-30 nucleobases in length, in still more embodiments, the morpholinos are at least about 30-35 nucleobases in length or more. The strengths of morpholinos as tools for investigating vertebrate development are well described in a recent review by Ekker S. C. (2000) Yeast 17:302-306, the disclosure of which is hereby incorporated by reference.

Morpholinos form RNA-morpholino hybrids that are not substrates for RNase H, and are not degraded. Ekker and colleagues report that fluorescently labeled morpholino oligonucleotides can be injected into sphere-stage zebrafish embryos and achieve uniform distribution. Morpholino oligomers targeted to the start codon for green fluorescent protein (GFP) blocked GFP expression, whereas control oligomers that are complementary to GFP did not. This established the ability of morpholino oligomers to unambiguously block gene expression in a sequence-specific manner. Ekker and colleagues also reported inhibition of several endogenous zebrafish genes.

The present invention provides morpholino-modified oligonucleotides targeting the 5′untranslated region of a GNG2 polynucleotide or a splice site of a GNG2 polynucleotide. Such morpholino-modified oligonucleotides are effective in inhibiting the expression of GNG2 and interfere with angiogenesis in an animal treated with these morpholino-modified oligonucleotides.

Morpholinos are highly non-polar. Thus, modified or unmodified morpholino oligos, may be administered in combination with any known delivery vehicle/vector that facilitates delivery of morpholino oligos into cells/tissues.

E. Delivery of Nucleic Acids to Cells:

The nucleic acid constructs of the invention may be delivered by any means known in the art. In some embodiments, nucleic acid is delivered into a cell using ex vivo strategies. In other embodiments, nucleic acid is delivered into a cell using in vivo strategies.

In ex vivo gene therapy methods, the cells are removed from the host organism, such as a human, prior to experimental manipulation. These cells are then transfected with a nucleic acid in vitro using methods well known in the art. These genetically manipulated cells are then reintroduced into the host organism. Alternatively, in vivo gene therapy approaches do not require removal of the target cells from the host organism. Rather, the nucleic acid may be complexed with reagents, such as liposomes or retroviruses, and subsequently administered to target cells within the organism using known methods. See, e.g., Morgan et al. (1987) Science 237:1476, 1987; Gerrard et al. (1993) Nat. Genet. 3:180.

Several different methods for transfecting cells can be used for either ex vivo or in vivo gene therapy approaches. Known transfection methods may be classified according to the agent used to deliver a select nucleic acid into the target cell. These transfection agents include virus dependent, lipid dependent, peptide dependent, and direct transfection (“naked DNA”) approaches. Other approaches used for transfection include calcium co-precipitation and electroporation.

Viral approaches use a genetically engineered virus to infect a host cell, thereby “transfecting” the cell with an exogenous nucleic acid. Among known viral vectors are recombinant viruses, of which examples have been disclosed, including poxviruses, herpesviruses, adenoviruses, and retroviruses. Such recombinants can carry heterologous genes under the control of promoters or enhancer elements, and are able to cause their expression in vector-infected host cells. Recombinant viruses of the vaccinia and other types are reviewed by Mackett et al. (1994) J. Virol. 49:3; also see Kotani et al. (1994) Hum. Gene Ther. 5:19.

Non-viral vectors, such as liposomes, may also be used as vehicles for nucleic acid delivery in gene therapy. In comparison to viral vectors, liposomes are safer, have higher capacity, are less toxic, can deliver a variety of nucleic acid-based molecules, and are relatively nonimmunogenic. See Felgner, P. L. and Ringold, G. M., (1989) Nature 337:387-388. Among these vectors, cationic liposomes are the most studied due to their effectiveness in mediating mammalian cell transfection in vitro. One technique, known as lipofection, uses a lipoplex made of a nucleic acid and a cationic lipid that facilitates transfection into cells. The lipid/nucleic acid complex fuses or otherwise disrupts the plasma or endosomal membranes and transfers the nucleic acid into cells. Lipofection is typically more efficient in introducing DNA into cells than calcium phosphate transfection methods. Chang et al. (1988) Focus 10:66.

One known protein dependent approach involves the use of polylysine mixed with a nucleic acid. The polysine/nucleic acid complex is then exposed to target cells for entry. See, e.g., Verma and Somia (1997) Nature 389:239; Wolff et al. (1990) Science 247:1465.

“Naked DNA” transfection approaches involve methods where nucleic acids are administered directly in vivo. See U.S. Pat. No. 5,837,693 to German et al. Administration of the nucleic acid could be by injection into the interstitial space of tissues in organs, such as muscle or skin, introduction directly into the bloodstream, into desirable body cavities, or, alternatively, by inhalation. In these so called “naked DNA” approaches, the nucleic acid is injected or otherwise contacted with the animal without any adjuvants. It has been reported that injection of free (“naked”) plasmid DNA directly into body tissues, such as skeletal muscle or skin, can lead to protein expression. See Ulmer et al. (1993) Science 259:1745-1749; Wang et al. (1993) Proc. Nat. Acad. Sci. USA 90:4157-4160; Raz et al. (1994) Proc. Nat. Acad. Sci. USA 91:9519-9523.

Electroporation is another transfection method. See U.S. Pat. No. 4,394,448 to Szoka, Jr. et al. and U.S. Pat. No. 4,619,794 to Hauser. The application of brief, high-voltage electric pulses to a variety of animal and plant cells leads to the formation of nanometer-sized pores in the plasma membrane. DNA can enter directly into the cell cytoplasm either through these small pores or as a consequence of the redistribution of membrane components that accompanies closure of the pores.

2. Host Cells

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, vertebrate GNG2 polypeptide can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding vertebrate GNG2 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) vertebrate GNG2 polypeptide. Accordingly, the invention further provides methods for producing vertebrate GNG2 polypeptide using the host cells of the invention. In one embodiment, the method comprises culturing the host cell of invention (into which a recombinant expression vector encoding vertebrate GNG2 has been introduced) in a suitable medium such that vertebrate GNG2 polypeptide is produced. In another embodiment, the method further comprises isolating vertebrate GNG2 from the medium or the host cell.

3. Transgenic Animals

The host cells of the invention can also be used to produce nonhuman transgenic animals. For example, in one embodiment, a host cell of the invention is a fertilized oocyte or an embryonic stem cell into which vertebrate GNG2-encoding sequences have been introduced. Such host cells can then be used to create non-human transgenic animals in which exogenous vertebrate GNG2 sequences have been introduced into their genome or homologous recombinant animals in which endogenous vertebrate GNG2 sequences have been altered. Such animals are useful for studying the function and/or activity of vertebrate GNG2 and for identifying and/or evaluating modulators of vertebrate GNG2 activity. As used herein, a “transgenic animal” is a non-human animal, in some embodiments, the animal is a fish, in other embodiments, the animal is a mammal (e.g., a rodent such as a rat or mouse), in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, such as a mammal (e.g., mouse) in which an endogenous GNG2 gene has been altered by homologous recombination between the endogenous gene and an exogenous DNA molecule introduced into a cell of the animal, e.g., an embryonic cell of the animal, prior to development of the animal.

A transgenic animal of the invention can be created by introducing vertebrate GNG2-encoding nucleic acid into the male pronuclei of a fertilized oocyte, e.g., by microinjection, retroviral infection, and allowing the oocyte to develop in a pseudopregnant female foster animal. The vertebrate GNG2 cDNA sequence of SEQ ID NO: 1, SEQ ID NO: 7, SEQ ID NO: 11, SEQ ID NO: 15, SEQ ID NO: 56, SEQ ID NO: 58, SEQ ID NO: 59, or SEQ ID NO: 60 or an artificial construct can be introduced as a transgene into the genome of a non-human animal. In some embodiments, variants of the vertebrate GNG2 are introduced in which the vertebrate GNG2 lacks one or more domains, or has substitutions of wild-type sequences with homologs. Alternatively, a homologue of the vertebrate GNG2 gene, can be isolated based on hybridization to the vertebrate GNG2 cDNA (described further above) and used as a transgene. Intronic sequences and polyadenylation signals can also be included in the transgene to increase the efficiency of expression of the transgene. A tissue-specific regulatory sequence(s) can be operably linked to the vertebrate GNG2 transgene to direct expression of vertebrate GNG2 polypeptide to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the vertebrate GNG2 transgene in its genome and/or expression of vertebrate GNG2 mRNA in tissues or cells of the animals. A transgenic founder animal can then be used to breed additional animals carrying the transgene. Moreover, transgenic animals carrying a transgene encoding vertebrate GNG2 can further be bred to other transgenic animals carrying other transgenes.

To create a homologous recombinant animal, a vector is prepared which contains at least a portion of a vertebrate GNG2 gene into which a deletion, addition or substitution has been introduced to thereby alter, e.g., functionally disrupt, the vertebrate GNG2 gene. The vertebrate GNG2 gene can be the cDNA of SEQ ID NO: 1, SEQ ID NO: 7, SEQ ID NO: 11, SEQ ID NO: 15, SEQ ID NO: 56, SEQ ID NO: 58, SEQ ID NO: 59 or SEQ ID NO: 60. The vertebrate GNG2 may then be introduced into the genome of a vertebrate, or other vertebrate. In one embodiment, the vector is designed such that, upon homologous recombination, the endogenous vertebrate GNG2 gene is functionally disrupted (i.e., no longer encodes a functional protein; also referred to as a “knock out” vector).

Alternatively, the vector can be designed such that, upon homologous recombination, the endogenous vertebrate GNG2 gene is mutated or otherwise altered but still encodes functional protein (e.g., the upstream regulatory region can be altered to thereby alter the expression of the endogenous vertebrate GNG2 polypeptide). In the homologous recombination vector, the altered portion of the vertebrate GNG2 gene is flanked at its 5′ and 3′ ends by additional nucleic acid of the vertebrate GNG2 gene to allow for homologous recombination to occur between the exogenous vertebrate GNG2 gene carried by the vector and an endogenous vertebrate GNG2 gene in an embryonic stem cell. The additional flanking vertebrate GNG2 nucleic acid is of sufficient length for successful homologous recombination with the endogenous gene. Typically, several kilobases of flanking DNA (both at the 5′ and 3′ ends) are included in the vector. See e.g., Thomas et al. (1987) Cell 51:503 for a description of homologous recombination vectors. The vector is introduced into an embryonic stem cell line (e.g., by electroporation) and cells in which the introduced vertebrate GNG2 gene has homologously recombined with the endogenous vertebrate GNG2 gene are selected (see e.g., Li et al. (1992) Cell 69:915). Methods of making transgenic non-human animals are well-known in the art (for mice see Brinster et al. (1985) Proc. Nat. Acad. Sci. USA 82:4438-42; U.S. Pat. Nos. 4,736,866, 4,870,009, 4,873,191, 6,127,598; Hogan, B., MANIPULATING THE MOUSE EMBRYO, Cold Spring Harbor Laboratory Press, Cold Spring Harbor, N.Y., (1986); for homologous recombination see Capecchi (1989) Science 244:1288-1292; Joyner et al. (1989) Nature 338:153-156; for particle bombardment see U.S. Pat. No., 4,945,050; for Drosophila see Rubin and Spradling (1982) Science 218:348-53, U.S. Pat. No. 4,670,388; for transgenic insects see Berghammer A. J. et al. (1999) Nature 402:370-371; for zebrafish see Lin S. (2000) Methods Mol. Biol. 136:375-3830; for fish, amphibians and birds see Houdebine and Chourrout, (1991) Experientia 47:897-905; for rats see Hammer et al. (1990) Cell 63:1099-1112; for embryonic stem (ES) cells see TERATOCARCINOMAS AND EMBRYONIC STEM CELLS, A PRACTICAL APPROACH, E. J. Robertson, ed., IRL Press (1987); for livestock see Pursel et al. (1989) Science 244:1281-1288; for nonhuman animal clones see Wilmut, I. et al. (1997) Nature 385:810-813, PCT Publication Nos. WO 97/07668 and WO 97/07669; for recombinase systems for regulated transgene expression see, Lakso et al. (1992) Proc. Natl. Acad. Sci. 89:6232-6236; U.S. Pat. No. 4,959,317 (for cre.loxP) and O'Gorman et al. (1991) Science 251:1351-1355; U.S. Pat. No. 5,654,182 (for FLP/FRT). If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

4. Vertebrate Polypeptides

The invention also provides vertebrate GNG2 polypeptides. The GNG2 polypeptides, variants, fragments and antigenic portions thereof may be derived from any vertebrate species. In some embodiments, the GNG2 polypeptide is derived from mammals. In some embodiments, the GNG2 polypeptide is derived from mice or rats. In other embodiments, the GNG2 polypeptide is derived from humans. In other embodiments, the GNG2 polypeptide is derived from fish, such as the zebrafish. In some embodiments, the polypeptides have the amino acid sequence of SEQ ID NO: 2.

The terms “polypeptide,” “peptide,” and “protein” are used interchangeably herein. “Polypeptide” refers to a polymer of amino acids without referring to a specific length. Polypeptides of the invention include peptide fragments, derivatives, and fusion proteins. Peptide fragments preferably have at least about 10, 15, 20, 25, 30, 35, 40, 45, 50, 60, 70, 80, 90, or 100 amino acids. Some peptide fragments of the invention are biologically active. Biological activities include immunogenicity, ligand binding, and activity associated with the reference peptide. Immunogenic peptides and fragments of the invention generate an epitope-specific immune response, wherein “epitope” refers to an immunogenic determinant of a peptide and preferably contains at least three, five, eight, nine, ten, fifteen, twenty, thirty, forty, forty-five, or fifty amino acids. Some immunogenic peptides of the invention generate an immune response specific to that peptide. Polypeptides of the invention include naturally occurring and non-naturally occurring peptides. The term includes modified polypeptides (wherein examples of such modifications include glycosylation, acetylation, phosphorylation, carboxylation, ubiquitination, labeling, etc.), analogs (such as non-naturally occurring amino acids, substituted linkages, etc.), and functional mimetics. A variety of methods for labeling polypeptides are well known in the art and include radioactive isotopes such as ³²P or ³⁵S, ligands that bind to labeled antiligands (e.g., antibodies), fluorophores, chemiluminescent agents, enzymes, and antiligands.

As used herein, the term “amino acid” denotes a molecule containing both an amino group and a carboxyl group. In some embodiments, the amino acids are α-, β-, γ- or δ-amino acids, including their stereoisomers and racemates. As used herein the term “L-amino acid” denotes an α-amino acid having the L configuration around the α-carbon, that is, a carboxylic acid of general formula CH(COOH)(NH₂)-(side chain), having the L-configuration. The term “D-amino acid” similarly denotes a carboxylic acid of general formula CH(COOH)(NH₂)-(side chain), having the D-configuration around the α-carbon. Side chains of L-amino acids include naturally occurring and non-naturally occurring moieties. Non-naturally occurring (i.e., unnatural) amino acid side chains are moieties that are used in place of naturally occurring amino acid side chains in, for example, amino acid analogs. Amino acid substituents may be attached, for example, through their carbonyl groups through the oxygen or carbonyl carbon thereof, or through their amino groups, or through functionalities residing on their side chain portions.

The amino acid sequences are presented in the amino (N) to carboxy (C) direction, from left to right. The N-terminal α-amino group and the C-terminal β-carboxy groups are not depicted in the sequence. Amino acids are represented in the manner recommended by the IUPAC-IUB Biochemical Nomenclature Commission, or amino acids are represented by their three letters code designations.

The invention also contemplates the use of cell-permeable peptides. Cell permeable peptides have been designed to inhibit the function of various pathways including neuronal degeneration (Borsello T, and C. Bonny (2004) “Use of cell-permeable peptides to prevent neuronal degeneration” Trends Mol. Med. 10(5):239-44 and Abeta1-40 fibrillogenesis (Gordon D. J. et al. (2002) “Design and characterization of a membrane permeable N-methyl amino acid-containing peptide that inhibits Abeta1-40 fibrillogenesis” J. Pept. Res. 60(1):37-55). Methods for designing cell-permeable peptides are known in the art and are described for example in Du C. et al. (1998) “Conformational and topological requirements of cell-permeable peptide function” J. Pept. Res. 51(3):235-243. Cell permeable peptides derived from GNG2 and/or VEGF may also be used in the methods of the invention to inhibit angiogenesis in a subject and to treat angiogenesis-related diseases and angiogenesis-dependent tumors. In order to determine which peptides are useful for such purpose, the screening procedures described herein may be used to routinely screen for functional cell-permeable peptides.

5. Different Antibodies that Specifically Recognize Vertebrate GNG2

The invention provides antibodies (e.g., monoclonal and polyclonal antibodies, single chain antibodies, chimeric antibodies, bifunctional/bispecific antibodies, humanized antibodies, human antibodies, and complementary determining region (CDR)-grafted antibodies), including compounds which include CDR sequences which specifically recognize vertebrate GNG2, or fragments of vertebrate GNG2 wherein the epitope comprises at least a portion of the amino acid sequence of SEQ ID NO: 2, SEQ ID NO: 3, SEQ ID NO: 4, SEQ ID NO: 5, SEQ ID NO: 6, and/or SEQ ID NO: 57.

Antibody fragments, including Fab, Fab′, F(ab′)₂, and FV, are also provided by the invention. The term “specific for,” when used to describe antibodies of the invention, indicates that the variable regions of the antibodies of the invention recognize and bind vertebrate GNG2 exclusively (i.e., are able to distinguish vertebrate GNG2 from other different G protein y subunits by virtue of measurable differences in binding affinity, despite the possible existence of localized sequence identity, homology, or similarity between vertebrate GNG2 and other γ subunit family member). “High binding affinity” refers to binding affinities of greater than about 5×10⁻⁸ M, preferably between about 5×10⁻⁸ M and about 5×10⁻¹² M, in some embodiments the binding affinity is about 5×10⁻⁹ M to about 5×10⁻¹¹ M, in some embodiments the binding affinity is about 5×10⁻⁷ M to about 5×10⁻⁸ M, in some embodiments the binding affinity is about 5×10⁻⁸ M to about 5×10⁻⁹ M, in some embodiments the binding affinity is about 5×10⁻⁹ M to about 5×10⁻¹⁰ M, in some embodiments the binding affinity is about 5×10⁻¹⁰ M to about 5×10⁻¹¹ M.

It will be understood that specific antibodies may also interact with other proteins (for example, Staphylococcus aureus protein A or other antibodies in ELISA techniques) through interactions with sequences outside the variable region of the antibodies, and, in particular, in the constant region of the molecule. Screening assays to determine binding specificity of an antibody of the invention are well known and routinely practiced in the art. For a comprehensive discussion of such assays, see Harlow et al. (Eds.), ANTIBODIES: A LABORATORY MANUAL; Cold Spring Harbor Laboratory; Cold Spring Harbor, N.Y., 1988, Chapter 6. Antibodies that recognize and bind fragments of vertebrate GNG2 are also contemplated, provided that the antibodies are specific for vertebrate GNG2. Antibodies of the invention can be produced using any method well known and routinely practiced in the art.

For the production of polyclonal antibodies, various suitable host animals (e.g., rabbit, goat, mouse or other mammal) may be immunized by injection with the native polypeptide, or a synthetic variant thereof, or a derivative of the foregoing. An appropriate immunogenic preparation can contain, for example, recombinantly expressed vertebrate GNG2 polypeptide or a chemically synthesized vertebrate GNG2 polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against vertebrate GNG2 can be isolated from the mammal (e.g., from the blood) and further purified by well known techniques, such as protein A chromatography to obtain the IgG fraction.

The term “monoclonal antibody” or “monoclonal antibody composition,” as used herein, refers to a population of antibody molecules that contain only one species of an antigen binding site capable of immunoreacting with a particular epitope of vertebrate GNG2. A monoclonal antibody composition thus typically displays a single binding affinity for a particular vertebrate GNG2 polypeptide with which it immunoreacts. For preparation of monoclonal antibodies directed towards a particular GNG2 polypeptide, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., (1983) Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al. (1983) Proc Natl Acad Sci USA 80:2026-2030) or by transforming human B-cells with Epstein Barr Virus in vitro (see Cole, et al. (1985) In: MONOCLONAL ANTIBODIES AND CANCER THERAPY, Alan R. Liss, Inc., pp. 77-96).

According to the invention, techniques can be adapted for the production of single-chain antibodies specific to a vertebrate GNG2 polypeptide (see e.g., U.S. Pat. No. 4,946,778). In addition, methodologies can be adapted for the construction of Fab expression libraries (see e.g., Huse, et al. (1989) Science 246:1275-1281) to allow rapid and effective identification of monoclonal Fab fragments with the desired specificity for a GNG2 polypeptide or derivatives, fragments, analogs or homologs thereof. Non-human antibodies can be “humanized” by techniques well known in the art. See e.g., U.S. Pat. No. 5,225,539. Antibody fragments that contain the idiotypes to a GNG2 polypeptide may be produced by techniques known in the art including, but not limited to: (i) an F(ab′)₂ fragment produced by pepsin digestion of an antibody molecule; (ii) an Fab fragment generated by reducing the disulfide bridges of an F(ab′)₂ fragment; (iii) an Fab fragment generated by the treatment of the antibody molecule with papain and a reducing agent and (iv) Fv fragments.

Additionally, recombinant anti-vertebrate GNG2 antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PTC/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559); Morrison (1985) Science 229:1202-1207; Oi et al. (1986) BioTechniques 4:214; Jones et al. (1986) Nature 321:552-525; Verhoeyan et al. (1988) Science 239:1534; and Beidler et al. (1988) J. Immunol. 141:4053-4060.

In one embodiment, methodologies for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a zebrafish GNG2 polypeptide is facilitated by generation of hybridomas that bind to the fragment of a vertebrate GNG2 polypeptide possessing such a domain. Antibodies that are specific for an Ig-like domain within a vertebrate GNG2 polypeptide, or derivatives, fragments, analogs or homologs thereof, are also provided herein.

Anti-GNG2 antibodies may be used in methods known within the art relating to the localization and/or quantitation of a vertebrate GNG2 polypeptide (e.g., for use in measuring levels of the vertebrate GNG2 polypeptide within appropriate physiological samples, for use in diagnostic methods, for use in imaging the polypeptide, and the like). In a given embodiment, antibodies for vertebrate GNG2 polypeptides, or derivatives, fragments, analogs or homologs thereof, that contain the antibody derived binding domain, are utilized as pharmacologically-active compounds (hereinafter “therapeutics”).

An anti-GNG2 antibody (e.g., monoclonal antibody) can be used to isolate zebrafish GNG2 by standard techniques, such as affinity chromatography or immunoprecipitation. An anti-GNG2 antibody can facilitate the purification of natural GNG2 from cells and of recombinantly produced vertebrate GNG2 expressed in host cells. Moreover, an anti-GNG2 antibody can be used to detect vertebrate GNG2 polypeptide (e.g., in a cellular lysate) in order to evaluate the abundance and pattern of expression of the vertebrate GNG2 polypeptide. Anti-GNG2 antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include Iodine-125, Iodine-131, Sulfur-35 or tritium. In addition, the antibodies of the present invention may be conjugated to toxins such as radioisotopes, protein toxins and chemical toxins. Such toxins include, but are not limited to Lead-212, Bismuth-212, Astatine-211, Iodine-131, Scandium-47, Rhenium-186, Rhenium-188, Yttrium-90, Iodine-123, Iodine-125, Bromine-77, Indium-111, Boron-10, Actinide, ricin, adriamycin, calicheamicin, and 5-fluorouracil.

6. Pharmaceutical Compositions

The GNG2 nucleic acid molecules, GNG2 polypeptides (including cell permeable modified versions of the protein), and anti-GNG2 antibodies (also referred to herein as “active ingredients”) of the invention, and derivatives, fragments, analogs and homologs thereof, can be incorporated into pharmaceutical compositions in therapeutically effective amounts suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a GNG2 polypeptide or anti-GNG2 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

7. Methods of Modulating Angiogenesis

Angiogenesis-related diseases may be diagnosed and treated using the vertebrate GNG2 protein of the present invention as drug target for development of therapeutics. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. In some embodiments, a goal is to promote angiogenesis in the subject. Angiogenesis is important, for example, in wound healing. Thus GNG2 or its products can be used, without limitation, to promote wound healing, to promote endothelialization in vascular graft surgery, and to promote endothelialization to heal vascular damage following myocardial infarction.

The invention provides methods of modulating angiogenesis in an animal. In some embodiments, the method promotes angiogenesis. In some embodiments, the method inhibits angiogenesis.

(A) Pro-Angiogenesis

In some embodiments of the invention, angiogenesis is promoted in cells by increasing the expression of GNG2. In the method of the invention, nucleic acid sequences encoding GNG2 are administered to cells to promote increased expression of GNG2. The nucleic acid sequences may be any GNG2 (i.e., may be derived from any species) provided the GNG2 nucleic acid molecules encode GNG2 with biological activity. The polynucleotides encoding GNG2 may be part of an expression vector as described herein. Methods of introducing nucleic acids into cells and animals are known in the art. The polynucleotides that may be used that encode GNG2 include, but are not limited to SEQ ID NO: 1, SEQ ID NO: 7, SEQ ID NO: 1, SEQ ID NO: 13 and SEQ ID NO: 15. Other sequences include those encoding other species GNG2s, such as cow (SEQ ID NO: 56), mouse (SEQ ID NO: 58 and SEQ ID NO: 60), and human (SEQ ID NO: 59). The amount of GNG2 expression may be driven by the strength of the promoter, and tissue specific factors as selected for the appropriate system. Generally, the amount of expression should be such that angiogenesis is stimulated. In some embodiments, GNG2 alone is sufficient, however, in other embodiments, co-expression of a specific G protein □ subunit in addition to expression of GNG2 is used to activate a biological pathway. Thus, the invention also comprises the co-expression of GNG2 and G protein □ subunit for the promotion of angiogenesis. Co-expression of the two subunits may be achieved by any means known in the art. Nucleic acids encoding the two subunits may be incorporated into the same expression vector or may be present on separate expression vectors. Transfection of the vectors into cells may be simultaneous or sequential. Expression of the two subunits may be under the control of the same regulatory elements or different regulatory elements to allow controlled expression of one or both subunits. Alternatively, the expression of either or both subunits may be made to be constitutive.

(B) Anti-Angiogenesis

In some embodiments of the invention, angiogenesis is inhibited in cells by decreasing the expression of GNG2, or by inhibiting the function of GNG2. Angiogenesis may be inhibited through the use of compounds targeted against GNG2, such as chemical compounds including natural or synthetic small molecules, antisense GNG2 molecules or antibodies against GNG2. The polynucleotides that inhibit angiogenesis are those that bind to GNG polynucleotides and inhibit the expression of GNG2 or the correct splicing of GNG2 RNA. Examples of antisense molecules that inhibit GNG2 expression include, but are not limited to SEQ ID NO: 8 MO-gng2 (translation): 5′-gccatgaggctggcggttcaggc-3′ (SEQ ID NO: 8), GNG2 (sense): 5′-gcctgaaccgccagcctcatggc-3′ (SEQ ID NO: 9), MO-gng2 (antisense): 5′-gccatgaggctggcggttcaggc-3′ (SEQ ID NO: 10), and MO-GNG2 (splicing): 5′-tatgctctttctgacctttattctg-3′ (SEQ ID NO: 12).

The compounds that may be administered to inhibit the function of GNG2 include chemical compounds including natural or synthetic small molecules, polyclonal and monoclonal antibodies that specifically bind GNG2. The antibodies may be those that bind any known GNG2 provided that when administered to the subject, the antibodies specifically bind the GNG2 produced by the cells of the subject.

(C) Anti-Angiogenesis Combination Therapy

In other embodiments of the invention, angiogenesis is modulated by manipulating the expression and/or function of GNG2 in combination with modulating the expression and/or function of vascular endothelial growth factor (VEGF). In some embodiments, angiogenesis is stimulated by administering GNG2-encoding nucleic acid in combination with VEGF-encoding nucleic acid. The GNG2 and VEGF nucleic acids may be present in a single expression vector or on separate expression vectors. The VEGF and GNG2 nucleic acids may be administered simultaneously or separately. In other embodiments, angiogenesis is inhibited by administering a compound that decreases expression or function of GNG2 and a compound that decreases the expression or function of VEGF. In some embodiments, the compound that decreases the expression of GNG2 is an antisense oligonucleotide. In other embodiments, the compound that decreases the expression of GNG2 is a morpholino directed against GNG2. In some embodiments, the compound that decreases the expression of VEGF is an antisense oligonucleotide. In other embodiments, the compound that decreases the expression of VEGF is a morpholino directed against VEGF. In some embodiments, the compound that inhibits the function of GNG2 is an anti-GNG2 polyclonal or monoclonal antibody. In some embodiments, the compound that inhibits the function of VEGF is an anti-VEGF polyclonal or monoclonal antibody.

VEGF has been cloned and sequenced and many different VEGFs are known, including VEGF-A, VEGF-B, VEGF-C and VEGF-D. Any VEGF-encoding nucleic acid that produces a biologically active VEGF may be used. For example, but not by way of limitation, the VEGF-encoding nucleic acids that may be used include that for human VEGFs (SEQ ID NO: 35) (protein: SEQ ID NO: 36), human VEGF-B (SEQ ID NO: 37)(protein: SEQ ID NO: 38), human VEGF-C (SEQ ID NO: 45)(protein: SEQ ID NO: 46), human VEGF-D (SEQ ID NO: 47)(protein: SEQ ID NO: 48); mouse VEGF-A (SEQ ID NO: 41)(protein: SEQ ID NO: 42), mouse VEGF-B (SEQ ID NO: 39)(protein: SEQ ID NO: 40), mouse VEGF-C (SEQ ID NO: 49)(protein: SEQ ID NO: 50), mouse VEGF-D (SEQ ID NO: 51)(protein: SEQ ID NO: 52); zebrafish VEGF (SEQ ID NO: 43)(protein: SEQ ID NO: 44), and the like.

In some embodiments, VEGF expression and/or function is inhibited by administering antisense sequences. The antisense sequences have the general properties described as for GNG2 antisense sequences. In specific embodiments, for example, the antisense sequences have the sequence of gtatcaaataaacaaccaagttcat (SEQ ID NO: 53), gtaacaattaaacaaccatgttgat (SEQ ID NO: 54), and taagaaagcgaagctgctgggtatg (SEQ ID NO: 55). SEQ ID NO: 53 and SEQ ID NO: 55 antisense sequences target zebrafish VEGF. The human VEGF sequence is found under accession number: NM_{—}003376, Unigene cluster: Hs.73793 (Ensembl gene ID: ENSG00000112715).

In some embodiments, VEGF function is inhibited by the administration of anti-VEGF polyclonal or monoclonal antibodies. Examples of anti-VEGF monoclonal antibodies include, AVASTIN™ (Genentech), and BioCarta Catalog Nos. 09-06-16460 and 09-06-11335. The VEGF signaling pathway may also be inhibited by any tyrosine kinase inhibitor known in the art to disrupt VEGF signaling, or any other type of inhibitor for VEGF known in the art. These inhibitors may be used in combination with strategies described herein to modulate expression or activity of GNG2.

In embodiments to inhibit the function of GNG2 and VEGF, the compounds that are used to inhibit the function, or biological activity, include, small molecule compounds, polynucleotides (e.g., antisense and morpholinos, ribozymes, and the like); antibodies against VEGF and GNG2; cell permeable peptides that block the interaction of GNG2 with other receptors (upstream receptors), G protein partners, and downstream effectors; and combinations of these approaches.

Screening for Compounds that Promote or Inhibit Angiogenesis:

Another aspect of the present invention is directed to methods of identifying compounds that bind to either GNG2 or nucleic acid molecules encoding GNG2, comprising contacting GNG2, or a nucleic acid molecule encoding GNG2, with a test compound, and determining whether the test compound binds GNG2 or a nucleic acid molecule encoding GNG2.

Binding can be determined by any binding assays known in the art, including but not limited to, gel-shift assays, Western blots, radiolabeled competition assay, phage-based expression cloning, co-fractionation by chromatography, co-precipitation, cross-linking, interaction trap/two-hybrid analysis, southwestern analysis, ELISA, and the like, which are described in, for example, CURRENT PROTOCOLS IN MOLECULAR BIOLOGY, 1999, John Wiley & Sons, NY. The GNG2 polypeptide or polynucleotide employed in such a test may either be free in solution, attached to a solid support, or located intracellularly, or associated with a cell fraction. In one embodiment of the invention, high throughput screening (“HTS”) for compounds having suitable binding affinity to GNG2 is employed. Large numbers of test compounds may be exposed to immobilized GNG2. Bound GNG2 is then detected by methods well known in the art.

Another method for identifying ligands of a target protein is described in Wieboldt et al., Anal. Chem., 69:1683-1691 (1997). This technique screens combinatorial libraries of 20-30 agents at a time in solution phase for binding to the target protein. Agents that specifically bind to the target protein which are retained on the filter are subsequently liberated from the target protein and analyzed by HPLC and pneumatically assisted electrospray (ion spray) ionization mass spectroscopy. This procedure selects library components with the greatest affinity for the target protein, and is particularly useful for small molecule libraries.

Other embodiments of the invention comprise using antibody-based competitive screening assays. In one embodiment, specific, neutralizing antibodies that bind GNG2 specifically compete with a test compound for binding to GNG2. In this manner, the antibodies can be used to detect the presence of any peptide that shares one or more antigenic determinants with GNG2. Such binding studies may use labeled antibodies and/or labeled test compounds. An examples of such a procedure can be found in, for example, Lin, A. H. et al. (1997) Antimicrobial Agents and Chemotherapy 41(10):2127-2131.

Another aspect of the present invention is directed to methods of identifying compounds that modulate (i.e., increase or decrease) the biological activity of GNG2. Such methods comprise contacting GNG2 with a test compound, and determining whether the compound affects the biological activity of GNG2 in a positive (agonist) or negative (antagonist) way as compared to the activity of GNG2 in the absence of the test compound.

In some embodiments, the compounds that modulate GNG2 expression or biological activity may be identified in an in vitro cell assay in which a test compounds is incubated with a cell expressing a GNG2 polypeptide or having a GNG2 polynucleotide and determining the effect the test compound has on GNG2 expression or biological activity. Modulators of GNG2 activity will be therapeutically useful in treatment of diseases involving angiogenesis. Compounds identified as modulating GNG2 expression of biological activity in vitro may be further tested vivo to confirm relevant and effective activity.

The invention also provides methods for identifying a GNG2 modulator by contacting a G protein β subunit and GNG2 in the presence or absence of a test compound and detecting an effect on the binding of the β subunit and GNG2. Agonists are those compounds that promote the binding of the β subunit and GNG2, while antagonists are those tests compounds that inhibit the binding of the β subunit and GNG2. These test compounds may be further tested in other in vivo assays to confirm relevant and effective activity.

Test compounds contemplated by the invention include compounds from chemical libraries, including natural products and/or synthetic products from combinatorial chemical synthesis. Such compounds may include random peptides, oligonucleotides, or organic molecules.

In some embodiments, transgenic zebrafish carrying a GNG2 promoter driven-GFP transgene are used to detect whether a compound, small molecule or gene product has regulatory activity on the promoter of the GNG2 gene, resulting in altered expression of the GFP. Increased or decreased activity will be detected by the fluorescent signal generated within the living zebrafish, thus provide an in vivo model for screening biologically active drugs, small molecules and gene products.

The compounds identified by the screening methods may be used in the methods of the invention to promote or inhibit angiogenesis, alone or in combination with the compounds that promote or inhibit VEGF.

The following examples are merely illustrative of the invention and are not to be construed as limiting the invention in any way.

### EXAMPLES

### Example 1

Fish Stocks: Danio rerio: Florida wildtype strain (Lancaster, Pa.) and Longfin strain (Scientific Hatcheries, Huntington Beach, Calif.).

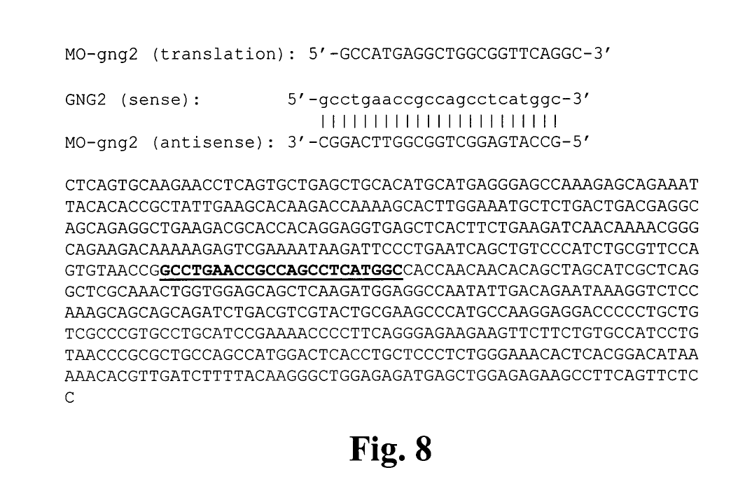

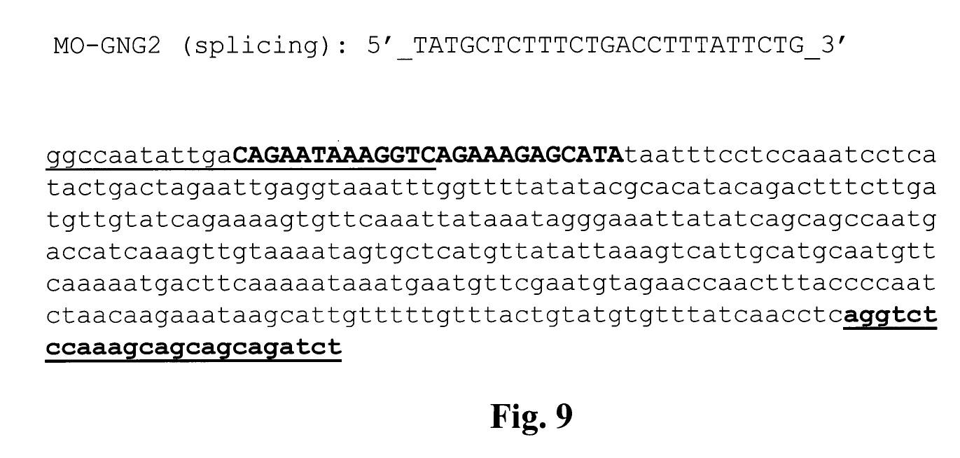

Zebrafish antisense GNG2 morpholino oligonucleotides (synthesized from GeneTools, LLC, Philomath, Oreg.) had the following sequences: Translation inhibition antisense: 5′-gccatgaggctggcggttcaggc-3′ (SEQ ID NO: 8) (See FIG. 8); Splicing antisense: 5′-tatgctctttctgacctttattctg (SEQ ID NO: 12) (See FIG. 9).

Microinjection of mRNA and Morpholino Antisense Oligo:

The VEGF mRNA overexpression construct was kindly provided by Dr. Brant Weinstein (Lawson, N. D. et al. (2003) Genes Dev. 17(11):1346-1351). Capped sense RNA was synthesized using SP6 RNA polymerase and the MMESSAGE mMACHINE system (Ambion). For microinjection of VEGF mRNA or morpholino antisense oligos, zebrafish embryos were injected with 0.25 to 0.5 nl into the yolk at the early one-cell stage, subsequently incubated in 0.3× Danieau's medium at 28.5° C. Embryos were maintained in the above condition until they had reached bud stage or 24 hour-post-fertilization and then collected for total RNA preparation or fixed in 4% paraformaldehyde for whole-mount in situ hybridization.

In Situ Hybridization:

The flk in situ construct was kindly provided by Dr. Brant Weinstein (Lawson, N. D. et al. (2003) Genes Dev. 17(11):1346-1351). The in situ hybridization procedure was modified from Leung, T. et al. (2003) Development 130(16):3639-3649. In brief, embryos were hybridized at 68° C. overnight, then washed by 66% Hyb/33% 2×SSC for 30 minutes, then washed by 33% Hyb/66% 2×SSC for 30 minutes at 68° C., then by 2×SSC for 15 minutes at 68° C., followed by 0.2×SSC for 1 hour at 68° C. The hybridized probes were detected by NBT/BCIP staining (Roche). After color staining, the embryos were washed in 100% ethanol for 1 hour.

RT-PCR:

Trizol (Invitrogen Life Technologies) was used to isolate total RNA from 10 uninjected (wildtype) embryos and 10 embryos injected with 100 μM morpholino antisense oligo (SEQ ID NO: 10). The RNA was treated with RQ1 DNase and 1 μg was reverse transcribed using MMLV Reverse Transcriptase (Promega). PCR was performed on the cDNA using Titanium Taq (BD Biosciences) for 30 and 26 cycles with GNG2 primers and 28S ribosomal subunit primers, respectively. GNG2 primers were as follows: Forward: 5′-atcgatatggccaccaacaacacagcta-3′ (SEQ ID NO: 31) and Reverse: 5′-ttacaggatggcacagaagaac-3′ (SEQ ID NO: 32). The 28S subunit primers were as follows: Forward: 5′-cctcacgatccttctggctt-3′ (SEQ ID NO: 33) and Reverse: 5′-attctgcttcacaatgata-3′ (SEQ ID NO: 34). Plasmids containing GNG2 genomic sequence or cDNA sequence were amplified as positive controls. The PCR products were run on a 2% agarose gel containing ethidium bromide.

Detection of MO Blocking Function by In Vitro System

The efficiency and specificity of the MO were assessed in an in vitro transcription and translation coupled system (TNT Coupled Reticulocyte Lysate Systems, Promega). The BamHI/XhoI gng2 PCR fragment was cloned into pcDNA3.1/V5-His-TOPO (Invitrogen). 0.5 μg of gng2 cDNA was used as template and different MO oligos were added. Western blotting was performed using anti-V5 antibody (Invitrogen). The immunoreactive proteins were detected by enhanced chemiluminescence (Amersham Pharmacia).

Whole-Mount Immunostaining

Embryos were fixed in 4% paraformaldehyde overnight, washed in PBST and blocked in PBST containing 2% goat serum and 2% BSA for 2 hours at 4° C. Primary antibodies against phospho-AKT and phospho-PLCγ1 (Cell Signaling Technology) were used in 1:100 dilution in blocking buffer at 4° C. overnight. After washing in PBST, secondary antibody were used in 1:300 dilution at 4° C. overnight. All above solutions contain 20 mM sodium fluoride and 2 mM sodium orthovanadate (Sigma). Color detection was done by DAB staining (Sigma).

Identification of the Zebrafish gng2 Gene

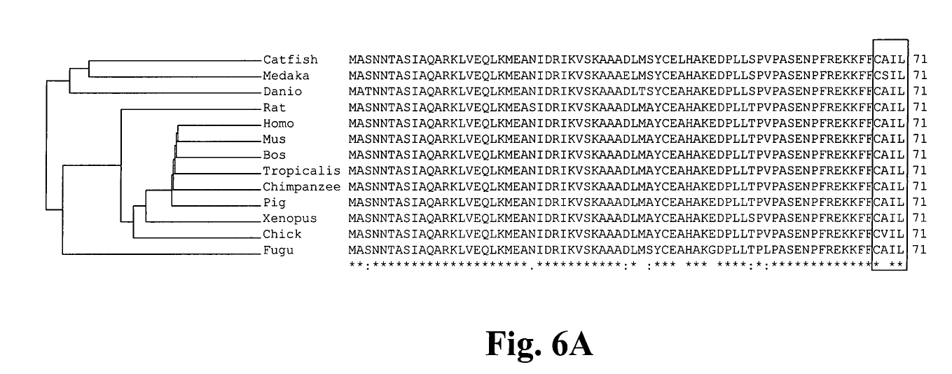

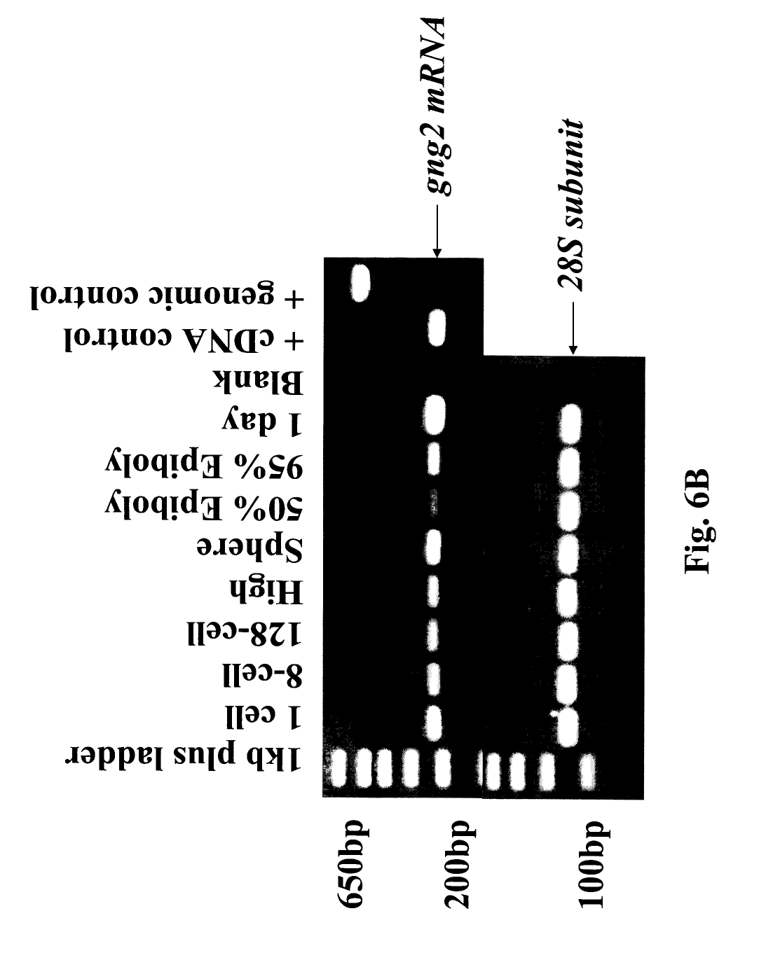

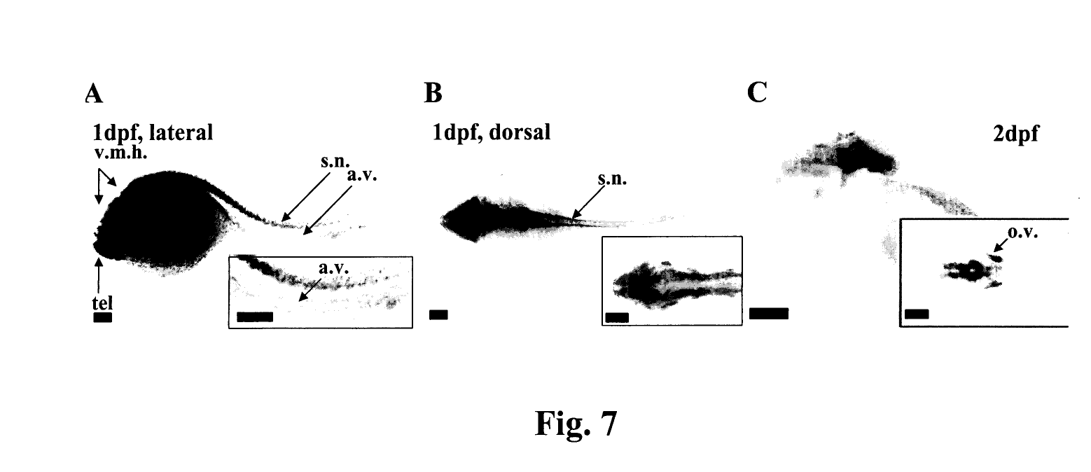

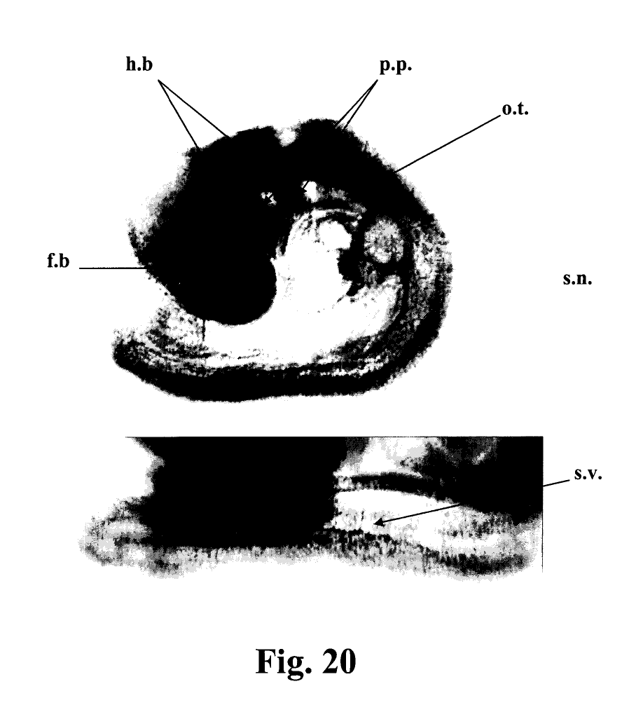

As a first step, we sought to identify those Gγ subtypes specifically expressed in the developing vasculature of the zebrafish embryos. Using the human and mouse G protein sequences to probe the zebrafish sequence database, we identified the zebrafish gng2 gene, which encodes the 71 amino acid Gγ2 protein that is highly conserved among vertebrates (FIG. 2 and FIG. 6A). The expression pattern of zebrafish gng2 was very dynamic during embryonic development. RT-PCR analysis showed the gng2 transcript was provided maternally from the 1-cell stage (FIG. 6B) and detected zygotically throughout gastrulation and embryogenesis. Whole-mount in situ hybridization using an antisense probe revealed that gng2 was expressed ubiquituously during gastrulation (data not shown). Subsequently, the transcript was spatially restricted to the central nervous system (CNS), including the telencephalon, ventral diencephalon, ventral hindbrain and spinal cord neurons, during morphogenesis. Besides the CNS, the transcript was also expressed in the axial vasculature including the dorsal aorta at 1 day-post-fertilization (dpf) although there was no detectable signal along the inter-somitic vessels (FIG. 7A and FIG. 7B). At 2 dpf, the transcript was down-regulated in the vasculature although it was still detected in other tissues such as the ventral midbrain, hindbrain, and otic vesicle (inner ear) (FIG. 7C). At 3 dpf, the transcript was almost undetectable by whole-mount in situ hybridization (data not shown). To compare the expression of gng2 with its mammalian counterpart, we also performed whole-mount in situ hybridization of E8.5 and E9.5 mouse embryos. During this stage of mouse embryonic development, the axial vasculature is being established and the intersomitic vessels are undergoing angiogenic sprouting from the dorsal aorta. Consistent with a possible role in this process, our results showed that the mouse gng2 transcript was detected in the vasculature and the sprouting vessels at E9.5 stage embryos (FIG. 20). This developmental stage of the angiogenic process is very similar to 1 dpf zebrafish embryos. Thus, the conserved protein sequence and similarity in expression patterns reinforces the idea that the zebrafish Gγ2 is a true ortholog of the mouse protein.

Targeted Knockdown of Zebrafish gng2 Inhibits Angiogenesis

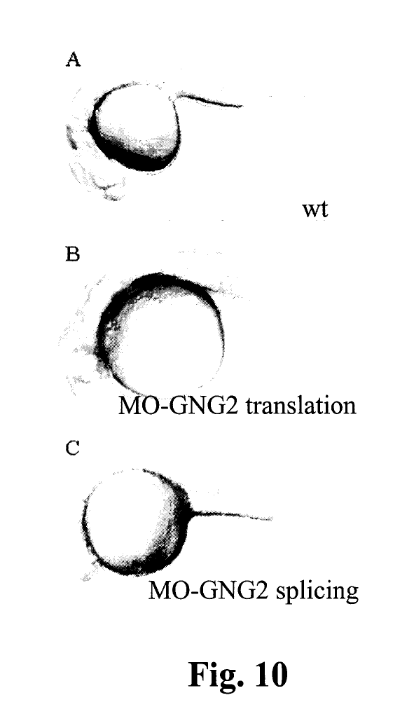

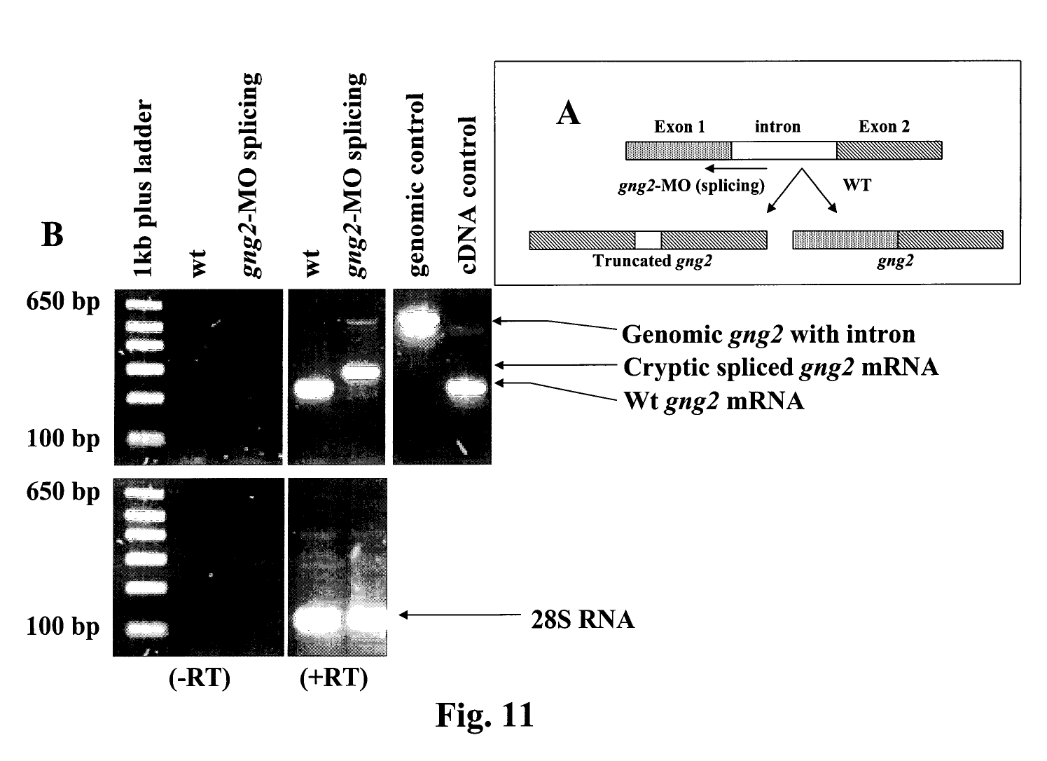

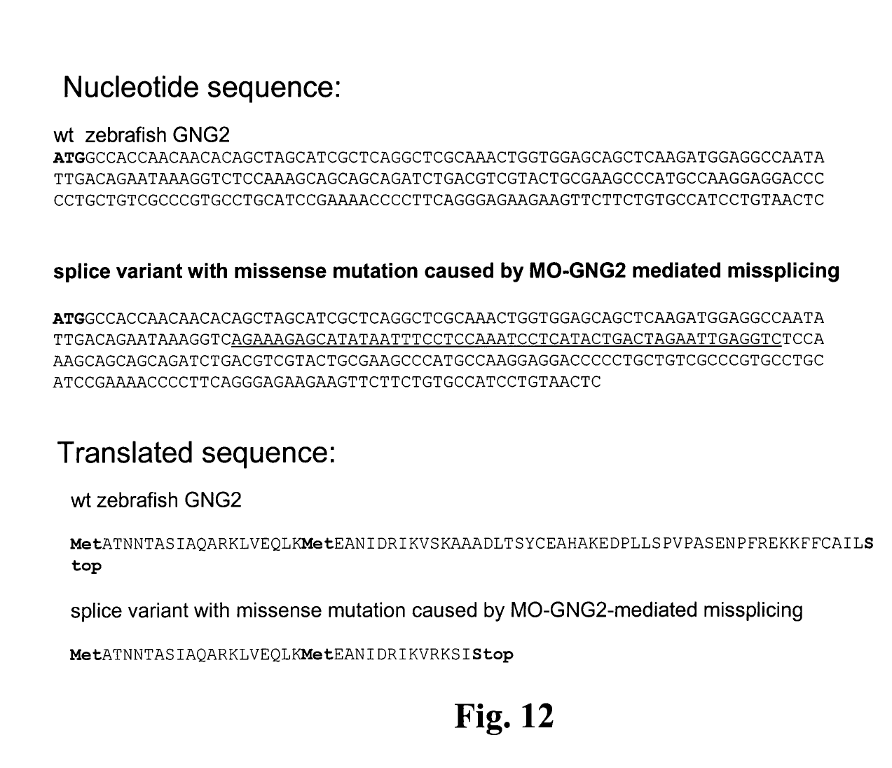

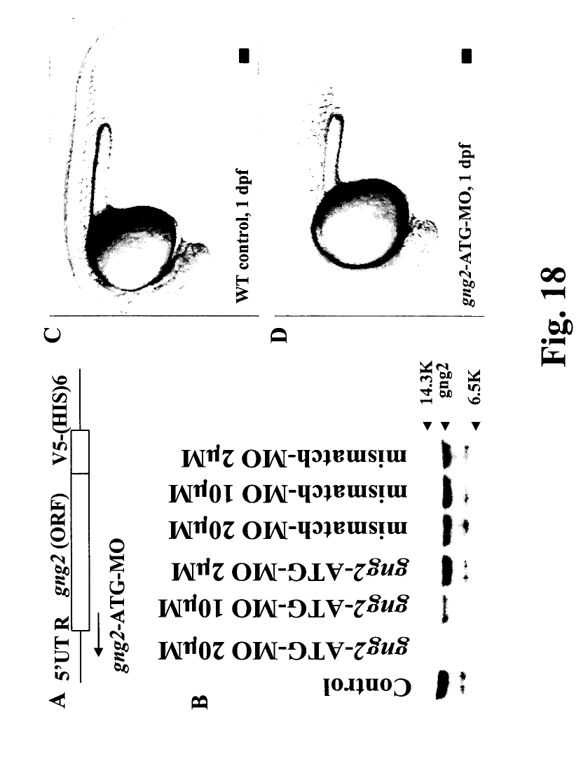

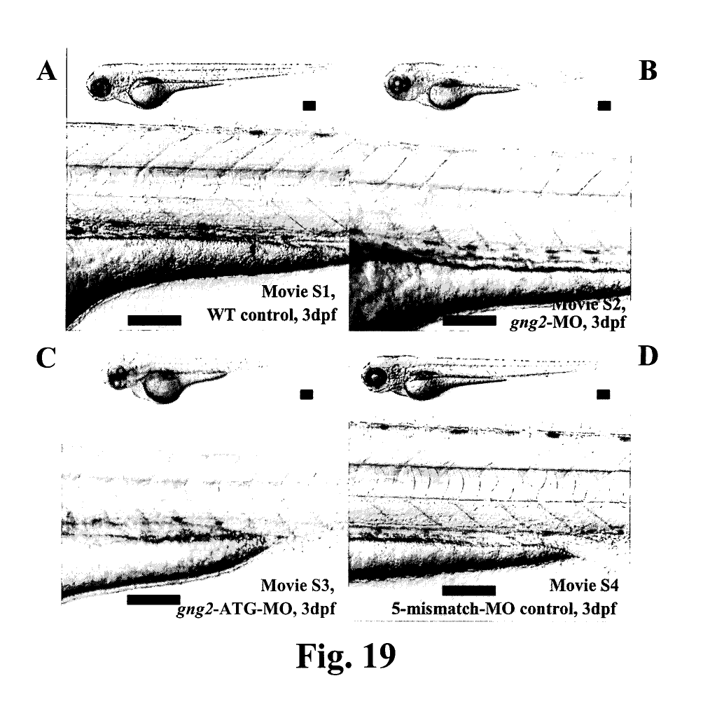

In order to uncover a possible role of gng2 in angiogenesis, we used the morpholino antisense knockdown approach in the zebrafish embryos (Nasevicius A. and SC Ekker (2000) Nat. Genet. 26:216-220). To demonstrate the efficacy of inhibition in vivo, we designed a splice junction morpholino targeted against the first coding exon-intron boundary (FIG. 11A). When injected into zebrafish embryos, the splicing morpholino induced a cryptic splicing site resulting in truncation of the coding sequence of gng2, as confirmed by sequencing of the RT-PCR products (FIG. 11B and FIG. 12). The first morphological sign of an angiogenic defect was detected at 3 dpf when the knockdown embryos showed reduced or missing blood flow through the inter-somitic vessels, although there was blood circulation in the dorsal aorta and cardinal vein (FIG. 19, Movie S1, WT; Movie S2, gng2-MO splicing knockdown). The specificity of this knockdown approach was confirmed using a second morpholino directed against the 5′UTR spanning the ATG start codon to inhibit translation (FIG. 18A; FIGS. 18C and D; FIG. 19; Movie S3). The translation blocking morpholino specifically inhibited Gγ2 protein expression, as validated by the in vitro translation assay (FIG. 18B). Both morpholino strategies produced a similar blood circulation phenotype in vivo, which was not observed in embryos injected with a 5-base mismatch control morpholino (FIG. 19, Movie S4, mismatch control morpholino). Therefore, using two independent morpholinos and a mismatch control, we demonstrated that the gng2 gene was specifically targeted and that targeted knockdown of gng2 disrupted the sprouting of inter-somitic vessels from the axial vasculature of the zebrafish embryos (See also FIG. 10).

These inter-somitic vessels are particularly interesting because they develop by angiogenic sprouting from the dorsal aorta and cardinal vein, a process that closely resembles tumor angiogenesis (Bergers G, and LE Benjamin (2003) Nature Rev. Cancer 3:401-410).

Molecular Analysis of gng2 Knockdown Phenotype

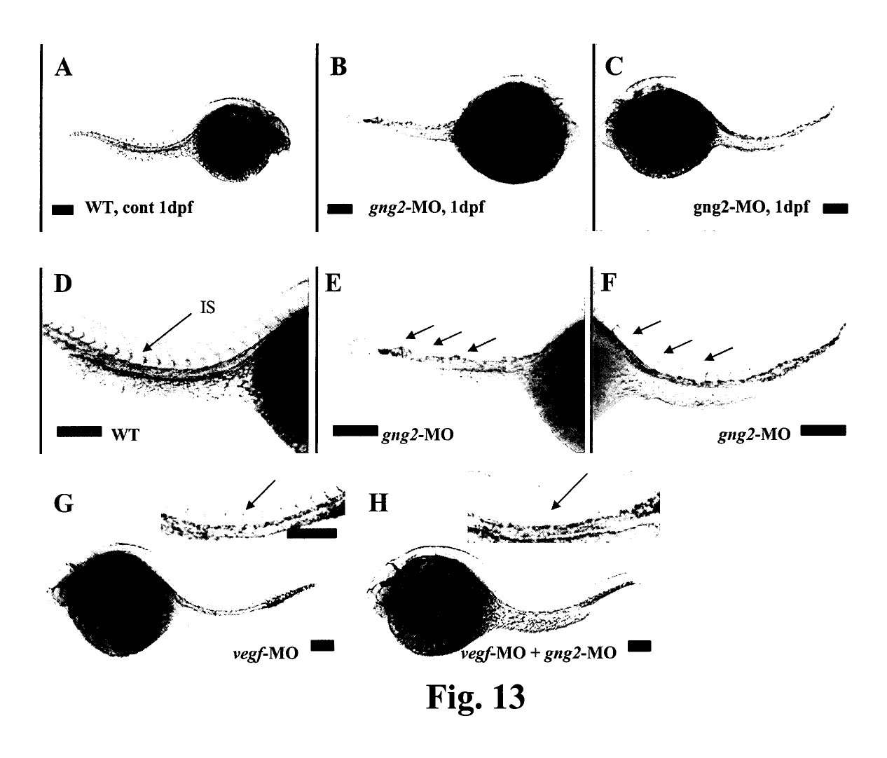

To examine the nature of the angiogenic defect in gng2 knockdown embryos, we used the endothelial specific marker flk1, which encodes the VEGFR-2 receptor (Flk-1/KDR), to visualize the developing vasculature (Liao W et al. (1997) Development 124:381-389). As shown by in situ hybridization of 1 dpf control embryos, the flk1 transcript was abundantly expressed in the cranial vessels, axial vasculature (including the dorsal aorta and cardinal vein), and more importantly, in the inter-somitic vessels sprouting from the dorsal aorta (FIG. 13A, and FIG. 13D). In situ hybridization analysis of gng2 knockdown embryos at the same stage revealed expression of the flk1 transcript along the dorsal aorta and cardinal vein, indicating that endothelial cell differentiation along the axial vasculature was not defective. However, gng2 knockdown embryos showed greatly reduced staining of the inter-somitic vessels sprouting from the axial vasculature (FIGS. 13B-C, FIGS. 13E-F; higher magnification in FIGS. 13E-F), suggesting that the defect was specific to the angiogenic process. Quantitative analysis revealed that 96% of the gng2 knockdown embryos exhibited partial (FIG. 13C and FIG. 13F) to complete loss of inter-somitic vessels (FIG. 13B and FIG. 13E), compared to the control embryos, all of which showed normal, and well established intersomitic vessels sprouting from the dorsal aorta (FIG. 13A and FIG. 13D). This phenotype persisted even after 30 hours-post-fertilization (data not shown), suggesting an actual defect rather than a delay in establishing the inter-somitic vessels in the gng2 knockdown embryos. Our finding that gng2 plays a critical role in angiogenesis provides an entry point for the study of G protein signaling in this process at the in organismo level. Furthermore, it demonstrates that a specific function for the Gγ2 subtype in vivo that cannot be substituted by other family members. Future studies will probe whether the failure of other Gγ subtypes to substitute for this function is due to its characteristic expression pattern or to its unique function in the context of the organism.

gng2 Genetically Interacts with the vegf Pathway

The gng2 knockdown phenotype shared striking similarity with that of vegf knockdown embryos (Nasevicius A. et al. (2000) Yeast 17:294-301; Childs S. et al. (2002) Development 129:973-982), suggesting they may function in a common or converging pathway. To explore this possibility, we compared the angiogenic response to vegf in control and gng2 suppressed embryos. Interestingly, morpholino knockdown of both the vegf and gng2 transcripts revealed a synergistic effect on angiogenesis in the zebrafish embryos (FIGS. 13G-H). At a sub-effective dosage of either morpholino alone, there was no effect on angiogenesis (FIG. 13G, vegf knockdown; gng2 knockdown data not shown). However, the simultaneous inhibition of both vegf and gng2 at the same sub-effective dose significantly eliminated the process of angiogenic sprouting in the zebrafish model (FIG. 13H).

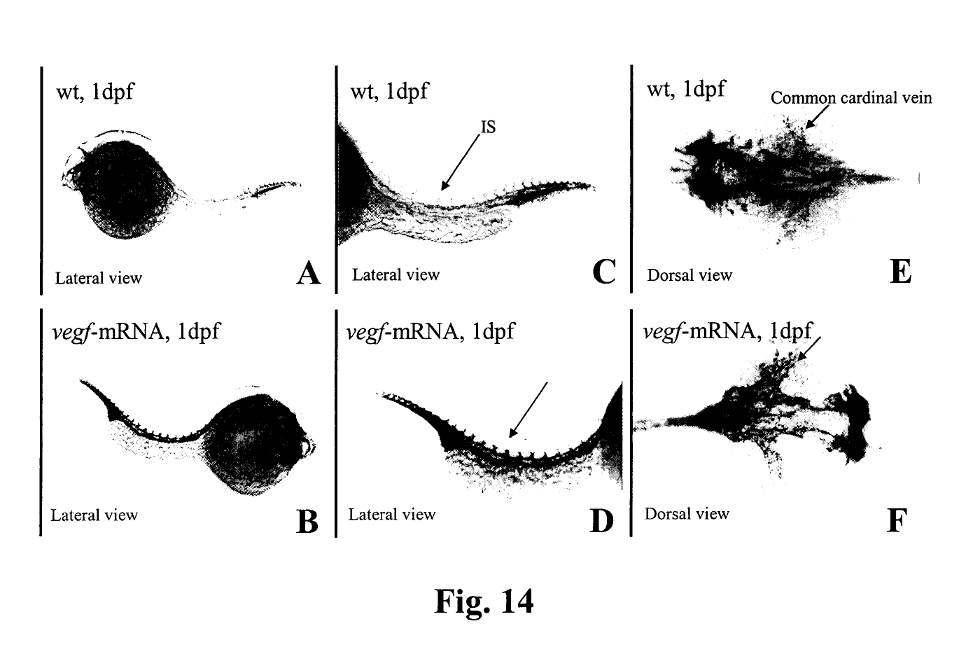

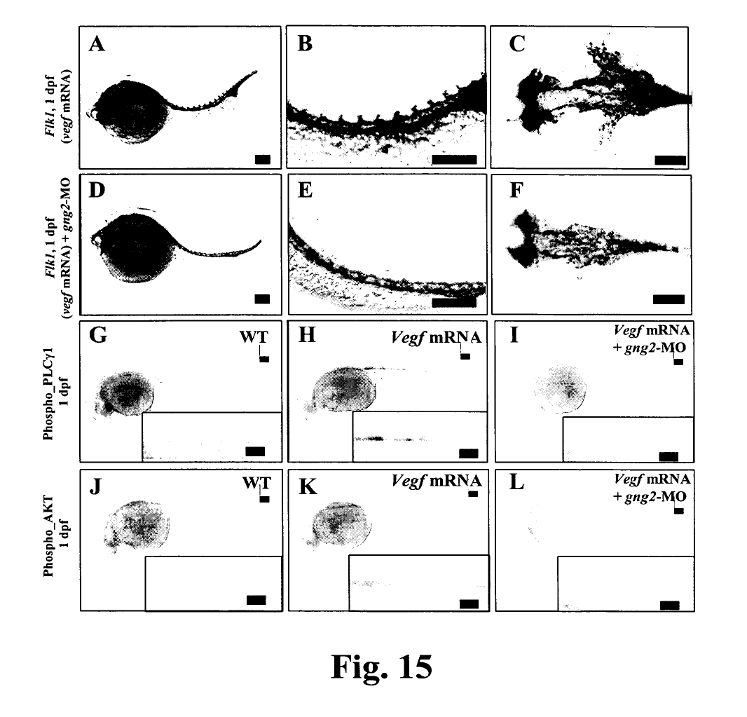

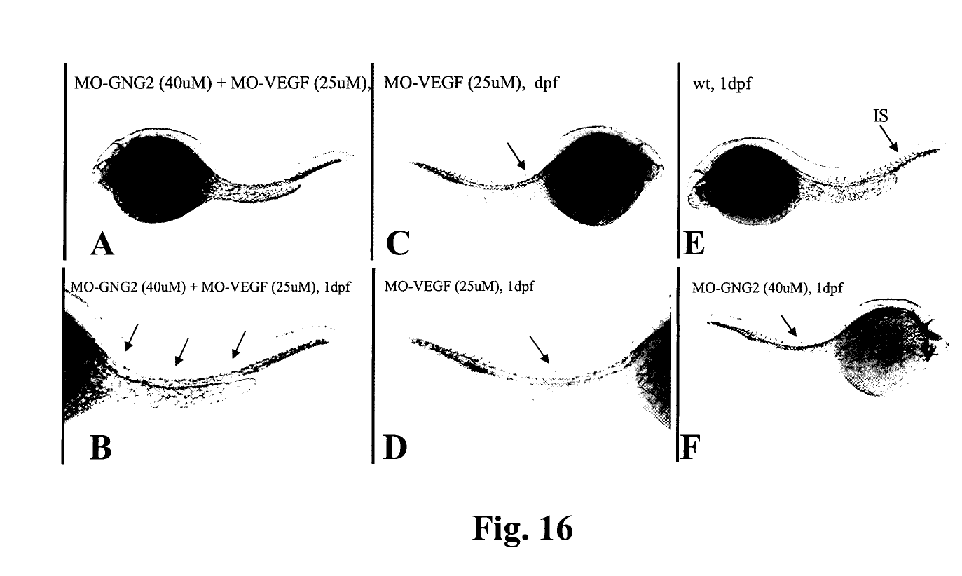

In another experiment, over-expression of vegf mRNA was used to induce endothelial cell proliferation, tube morphogenesis, and increased expression of flk1 in the axial vasculature and the inter-somitic vessels of developing zebrafish embryos (FIGS. 15A-B). Despite its pro-angiogenic properties, vegf over-expression followed by morpholino knockdown of gng2 specifically inhibited the inter-somitic vessels sprouting from the dorsal aorta (FIGS. 15D-E). More strikingly, the sprouting process from the dorsal aorta to the yolk common cardinal vein was completely inhibited by gng2 suppression even after vegf over-expression (FIG. 15F, vegf mRNA followed by gng2-MO; FIG. 15C, vegf mRNA alone). Taken together, these results show that gng2 genetically interacts with the vegf pathway and that loss of gng2 suppresses the pro-angiogenic effect of vegf (See also FIGS. 14 and 16).

G Protein γ2 Function is Crucial for VEGF Dependent Activation of PLCγ1 and AKT

Activation of the VEGFR-2 receptor (Flk-1/KDR) stimulates AKT kinase and PLCγ1 signaling molecules in vascular endothelial cells (Tanimoto T. et al. (2002) J. Biol. Chem. 277:42997-43001; Claesson-Welsh L. (2003) Biochem. Soc. Trans. 31:20-24; Takahashi T. et al. (2001) EMBO J. 20:2768-2778). To dissect the molecular pathway by which Gγ2 interacts with VEGFR-2 during angiogenesis, we used specific anti-phospho antibodies to detect the active forms of AKT kinase and PLCγ1 in the vasculature of developing zebrafish embryos. Upon over-expression of vegf mRNA in zebrafish embryos, we detected an activation of PLCγ1 and AKT kinase in the axial vasculature by whole-mount immunohistochemistry (FIG. 15H, K, compare to WT in FIG. 15G and FIG. 15J). Notably, knockdown of gng2 abolished the vegf induced phosphorylation of both PLCγ1 and AKT kinase (FIG. 151 and FIG. 15L). Thus, we have shown that Gγ2 plays a crucial and specific role in angiogenesis by blocking downstream components of the vegf signaling pathway in vivo.

Essential Role of G Protein γ2 for VEGF Signaling During Angiogenesis

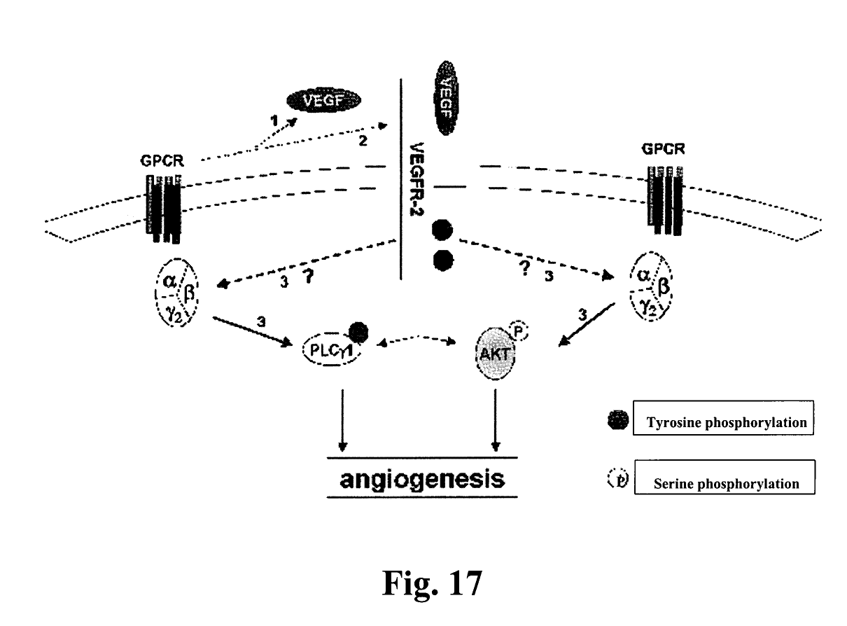

We have established the Gγ2 as a novel player during angiogenesis in the developing zebrafish embryos. In addition, we have shown that loss of gng2 blocks the ability of VEGF to promote angiogenic sprouting by disrupting downstream signaling to PLCγ1 and AKT. Without wishing to be bound by any particular theory of mechanism or operability, as shown in FIG. 17, we have considered three possible scenarios to explain the interaction between the Gγ2- and VEGF-dependent signaling pathways: 1) induction of VEGF expression; 2) transactivation of VEGFR-2 (Flk-1/KDR); and/or 3) direct coupling to VEGFR-2. Regarding the first possible scenario, several studies have shown that activation of GPCRs by thrombin, angiotensin, and endothelin can modulate vascular remodeling by inducing VEGF expression (Bagnato A, and F. Spinella (2003) Trends Endocrinol. Metab. 14:44-50; Richard D E et al. (2000) J. Biol. Chem. 275:26765-26771; Williams B. et al. (1995) Hypertension 25:913-917). However, this explanation seems unlikely in the current study since loss of gng2 produced a defect in vegf signaling even under conditions when vegf was over-expressed, and clearly, was not rate limiting.

Regarding the second possible scenario, some studies have found that activation of many of these same GPCRs can also transactivate the VEGFR-2 receptor by inducing its expression and/or stimulating its tyrosine phosphorylation (Tanimoto T. et al. (2002) J. Biol. Chem. 277:42997-43001; Miura S. et al. (2003) Hypertension 41:1118-1123; Maragoudakis M E et al. (2002) Biochemical Society Transactions 30:173-177; Imanishi T. et al. (2004) Hypertens Res. 27:101-108). Other studies have pointed to a similar involvement of G proteins in these processes (Zeng H. et al. (2003) J. Biol. Chem. 278:20738-20745; Albig A R and W P Schiemann (2005) Mol. Biol. Cell 16:609-625) with one report describing the physical interaction of Gαq/11 with Flk-1/KDR in human endothelial cells (Zeng H. et al. (2003) J. Biol. Chem. 278:20738-20745). However, the current study points to a different scenario since targeted knockdown of gng2 disrupted the sprouting of inter-somitic vessels from the axial vasculature even though expression of flk-1 along the dorsal aorta and cardinal vein was not affected.

In fact, ectopic over-expression of vegf mRNA in the gng2 knockdown embryos further induced expression of flk-1 along the dorsal aorta and cardinal vein even though the embryos still failed to develop inter-somitic vessels. Thus, the autoregulatory loop for VEGF and its receptor appeared to be intact in the gng2 knockdown embryos, making it less likely that Gγ2 interacts with VEGF signalling at the level of ligand or receptor expression. Regarding the third scenario, VEGFR-2 (Flk-1/KDR) belongs to the receptor tyrosine kinase superfamily. Some members of this superfamily have been shown to utilize G proteins for their downstream signaling. For example, the platelet-derived growth factor β receptor activates MAPK and DNA synthesis through direct tyrosine phosphorylation of Gαi (Alderton F. et al. (2001) J. Biol. Chem. 276:28578-28585); the insulin receptor stimulates phosphatidylinositol 3-kinase (PI3K) and glucose transport through activation of Gαq; 27 and the insulin like growth factor receptor activates MAPK and cell proliferation through interaction with Gαi (Kuemmerle J F et al. (2001) J. Biol. Chem. 276:7187-7194). Pointing to a similar requirement for a G protein in VEGFR-2 signaling, we showed that loss of Gγ2 attenuates the VEGF-induced activation of PLCγ1 and AKT during angiogenesis. We speculate that loss of Gγ2 disrupts the synthesis and assembly of the specific Gαγγ heterotrimer involved in this process. This possibility is consistent with recent evidence showing the Gγ component is required for stabilization and membrane trafficking of specific Gαβγ heterotrimers (Schwindinger W F et al. (2003) J. Biol. Chem. 278:6575-6579; Schwindinger W F et al. (2004) Mol. Cell. Biol. 24:7758-7768; Hynes T R et al. (2004) J. Biol. Chem. 279:30279-30286). Because receptor activation of the Gαβγ heterotrimer produces two active signaling moieties (Gα and Gγγ), its loss would have two consequences. First, signaling by the active Gβγ moiety would be blocked. In this regard, heterologous expression and reconstitution studies have shown that Gβγ can bind to and activate PLCγ1 in intestinal epithelial cells (Thodeti C K et al. (2000) J. Biol. Chem. 275:9849-9853). Similarly, Gβγ can activate p110 catalytic subunit of PI3K, which in turn activates AKT in the Chinese hamster ovary (CHO) cells (Suire S. et al. (2005) Curr. Biol. 15:566-570; Kubo H. et al. (2005) Biochem. J. August 10. [Epub ahead of print]). Although suggesting possible mechanisms, the specific combinations of Gγγ subunits involved in these processes were not determined in vivo. Based on the results of this current study, we speculate that a Gβγ dimer containing Gγ2 may be involved in activation of one or both of these enzymes in vascular endothelial cells. In addition to disruption of Gβγ mediated signaling, its loss would also block signaling by the Ga moiety since a specific Gαβγ heterotrimer is required for the activation by receptor (Schwindinger W F et al. (2001) Oncogene 20:1653-1660; Wang Q. et al. (1999) J. Biol. Chem. 274:17365-17371; Robillard L. et al. (2000) Cell Signal 12:673-682; Lim W K et al. (2001) Biochemistry 40:10532-10541). The identity of the Ga subunit associated with Gγ2 is not known. However, based on multiple lines of evidence, including similarity of knockout mice phenotypes, we speculate Gα13 or Gαq/11 may be involved (Offermanns S. et al. (1997) Science 275:533-536; Ruppel K M et al. (2005) Proc. Natl. Acad. Sci. USA 102:8281-8286; Offermanns S. et al. (1998) EMBO J. 17:4304-4312). This possibility will be explored in future studies.

### Example 2

Mouse gng2 in situ was performed using an RNA probe complementary to a fragment of the cDNA sequences (NM_{—}010315, 2962 bp mRNA, Mus musculus guanine nucleotide binding protein (G protein), gamma 2 subunit (Gng2), mRNA) (SEQ ID NO: 60).

ggagccaagc aagtcagatc tgccagggag cgtcaggcct tgaacactga ccagagtctc tgaagacccc
atccaacgct ccaatggcca gcaacaacac cgccagcata gcacaagcca ggaagctggt agaacagctg
aagatggaag ccaacatcga caggataaag gtgtccaagg cagctgctga cttgatggcc tactgtgagg
cacatgccaa ggaagaccct ctgctgaccc cagtcccagc ctcagaaaac ccctttcggg agaagaagtt
cttctgcgcc atcctttaag tctctgagag gaggctgaag agtgttgggg ctcctgggac atacatgtag
agttcctagc aaagtgggcg cctttctcgt ccacagcatt taaagagagg aaggagaacc atcctggaca
ctccgggctg tgcatgttta aagaaatgtc cccttatgag aatgaaagct gattccgtgt cccaacttta
gagatctgac cctgcagacc ggcctggagg agggaaatgt ataaaaaatg agaatggtaa tcacttcttt
tctgctgtcc ctctaagaca ttttctgctt catatttata aacaaaaata aaacatttaa aagcccggtg
ttcctggagc ctgagaatat gggctggaca tttctgggta atgagaaatt ggtcttttca aaatacccct
ctgataagct ggtcgccccc atgctagccc acaaatccaa gtttaatggg gtttccaacg tttaagtctt
ttggtgcctt tttcttttcc ttccctttct tctcttttag agggctggag aggataagac tgggtttgtg
tgttagtctc aactccaagc aggaatgaac cctgagatgt ttagaaaagt gttccccatg gggatttctt
catgagaata acaagaaaat agaaagaaat acatacatgt acacacacac acatgcacgc acacacacac
accacacaat tccggctctc tgtgttctct taccactatt cttaatattt gtatatgaca gttttgattc
tacgagttaa agtgaaccac gtgttgtgac tggtgcttcc atatctgtga tagtttgtga gtactattgc
atgaacatgt ccctacatcg catcc

The cDNA fragment was amplified from adult mouse brain cDNA using primers (underlined in the above sequence) for PCR: Mus_GNG2_{—}70F (5′-ggagccaagcaagtcagatc-3′) (SEQ ID NO: 61) and Mus_GNG2_{—}1194R (5′-ggatgcgatgtagggacatg-3′) (SEQ ID NO: 62). Mouse gng2 expression was detected in the forebrain, midbrain and hindbrain, including the eye anlagen and spinal cord neurons. Most importantly, it was also expressed in the developing intersomitic blood vessel of embryonic day 9.5 (E9.5) mouse embryos (FIG. 20). Thus the protein sequence and the expression pattern of gng2 are also conserved between fish and mammal. An exemplary reference for a standard mouse in situ protocol is Belo, J A, T. Bouwmeester, L. Leyns, N. Kertesz, M. Gallo, M. Follettie, and E M De Robertis (1997) “Cerberus-like is a secreted factor with neutralizing activity expressed in the anterior primitive endoderm of the mouse gastrula” Mech. Dev. 68(1-2):45-57.

What is claimed:

1. An isolated expression vector comprising a polynucleotide consisting of:
(a) SEQ ID NO: 1 and
(b) a sequence encoding a polypeptide having the amino acid sequence of SEQ ID NO: 2.

2. A host cell comprising the expression vector of claim 1.

3. An antibody that specifically binds to a polypeptide comprising the amino acid sequence of SEQ ID NO: 2 with high affinity, but which does not bind a polypeptide comprising the amino acid sequence of SEQ ID NO: 4 or SEQ ID NO: 5 with high affinity.

4. The antibody of claim 3, wherein said antibody is monoclonal.

5. The antibody of claim 3, wherein said antibody is polyclonal.

6. A method of producing a zebrafish GNG2 comprising culturing the host cell of claim 2 under conditions suitable for the expression of zebrafish GNG2, and isolating said GNG2.

7. A therapeutic composition comprising, an isolated polynucleotide comprising a nucleic acid sequence encoding zebrafish GNG2 and a pharmaceutically acceptable carrier.

8. The therapeutic composition of claim 7 wherein said nucleic acid sequence encodes the polypeptide of SEQ ID NO: 2.

9. A therapeutic composition comprising the antibody of claim 3 and a pharmaceutically acceptable carrier.

10. A therapeutic composition comprising a polypeptide comprising the amino acid sequence of SEQ ID NO: 2 and a pharmaceutically acceptable carrier.

11. A method of treating an angiogenesis-related disease comprising administering to a patient in need of such treatment a polynucleotide of that inhibits the expression of GNG2 in an amount sufficient to inhibit angiogenesis.

12. The method of claim 11, wherein the angiogenesis-related disease is selected from the group consisting of angiogenesis-dependent cancers; benign tumors; rheumatoid arthritis; psoriasis; ocular angiogenesis diseases; Osler-Webber Syndrome; myocardial angiogenesis; plaque neovascularization; telangiectasia; hemophiliac joints; angiofibroma; wound granulation; intestinal adhesions, atherosclerosis, scleroderma, hypertrophic scars, cat scratch disease and Helicobacter pylori ulcers.

13. The method of claim 11, wherein the angiogenesis-related disease is angiogenesis-dependent cancer.

14. The method of claim 11 wherein said angiogenesis-related disease is an angiogenesis-dependent tumor, and wherein said polynucleotide is administered in an amount sufficient to cause tumor regression.

15. A method of promoting angiogenesis in an animal in need thereof, comprising administering to an animal an effective amount of a polynucleotide encoding a GNG2 polypeptide.

16. The method of claim 15 wherein said GNG2 polypeptide comprises an amino acid sequence selected from the group consisting of SEQ ID NO: 2, SEQ ID NO: 4, SEQ ID NO: 5 and SEQ ID NO: 6.

17. The method of claim 15 wherein said GNG2 polypeptide comprises the amino acid sequence of SEQ ID NO: 2.

18. A method of treating an angiogenesis-related disease comprising administering to a patient in need of such treatment a first polynucleotide that inhibits the expression of GNG2 and a second polynucleotide that inhibits the expression of VEGF, wherein said first polynucleotide and said second polynucleotide are provided in an sufficient to inhibit angiogenesis.

19. The method of claim 18, wherein said first polynucleotide comprises a nucleic acid sequence selected from the group consisting of SEQ ID NO: 8, SEQ ID NO: 9, SEQ ID NO: 10, SEQ ID NO: 12 and combinations thereof.

20. The method of claim 18, wherein said second polynucleotide comprises a nucleic acid sequence selected from the group consisting of SEQ ID NO: 53, SEQ ID NO: 54, SEQ ID NO: 55 and combinations thereof.

21. The method of claim 18, wherein the angiogenesis-related disease is angiogenesis-dependent cancer.

22. A method of treating a patient with an angiogenesis-dependent tumor comprising administering to a patient in need of such treatment a first compound that inhibits the expression or function of GNG2 and a second compound that inhibits the expression or function of VEGF, wherein said first compound and said second compound are provided in amount sufficient to cause tumor regression.

23. A method of promoting angiogenesis in an animal in need thereof comprising administering to an animal an effective amount of a first polynucleotide encoding a GNG2 polypeptide and an effective amount of a second polynucleotide encoding a VEGF polypeptide.

24. The method of claim 23 wherein said GNG2 polypeptide comprises an amino acid sequence selected from the group consisting of SEQ ID NO: 2, SEQ ID NO: 4, SEQ ID NO: 5 and SEQ ID NO: 6.

25. The method of claim 23 wherein said VEGF polypeptide comprises an amino acid sequence selected from the group consisting of SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, and SEQ ID NO: 52.

26. The method of claim 23 wherein said GNG2 polypeptide comprises the amino acid sequence of SEQ ID NO: 2 and said VEGF polypeptide comprises the amino acid sequence of SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, and SEQ ID NO: 52.

27. The method of claim 22 wherein said second compound is an antibody that specifically binds VEGF.

28. A method of treating an angiogenesis-related disease comprising administering to a patient in need of such treatment a first compound that inhibits the function of GNG2 and a second compound that inhibits the function of VEGF, wherein said first compound and said second compound are provided in an sufficient to inhibit angiogenesis.

29. The method of claim 28 wherein said second compound is an antibody that specifically binds VEGF.

30. A method for identifying a compound that inhibits GNG2 activity comprising contacting a test compound with a GNG2 polypeptide and determining whether said test compound inhibits the activity of GNG2, wherein a test compound that inhibits the activity of GNG2 is identified as an antagonist of GNG2.

31. The method of claim 30 wherein said biological activity of GNG2 is measured by binding of said test compound to GNG2.

32. The method of claim 30 wherein said biological activity of GNG2 is measured by inhibition of binding of GNG2 to a β subunit.

33. The method of claim 30 wherein said biological activity of GNG2 is measured by inhibition of angiogenesis in a model system.

34. The method of claim 33 wherein said model system is a zebrafish development system.

35. The method of claim 33 wherein said model system is a transgenic animal system.

36. The method of claim 33 wherein said model system is an in vitro cell system.

37. A method of inhibiting angiogenesis comprising administering to a cell an effective amount of a cell permeable peptide that inhibits the biological function of GNG2.

38. The method of claim 37 further comprising the administration of a compound that inhibits the expression or biological function of VEGF.
